SEQUENCE LISTING (39 sequences)

SEQ ID NO: 1 - DNA, 743 bp, Homo Sapiens
  misc_feature: TSLP
  CDS (200)..(676)
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo Sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, Homo Sapiens
  MISC_FEATURE: LCDR1
GGNNLGSKSVH

SEQ ID NO: 4 - protein, 7 aa, Homo Sapiens
  MISC_FEATURE: LCDR2
DDSDRPS

SEQ ID NO: 5 - protein, 11 aa, Homo Sapiens
  MISC_FEATURE: LCDR3
QVWDSSSDHVV

SEQ ID NO: 6 - protein, 5 aa, Homo Sapiens
  MISC_FEATURE: HCDR1
TYGMH

SEQ ID NO: 7 - protein, 17 aa, Homo Sapiens
  MISC_FEATURE: HCDR2
VIWYDGSNKHYADSVKG

SEQ ID NO: 8 - protein, 13 aa, Homo Sapiens
  MISC_FEATURE: HCDR3
APQWELVHEAFDI

SEQ ID NO: 9 - DNA, 366 bp, Homo Sapiens
  misc_feature: Heavy Chain VH
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, Homo Sapiens
  MISC_FEATURE: Heavy Chain VH
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, Homo Sapiens
  misc_feature: Light Chain VL
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, Homo Sapiens
  MISC_FEATURE: Light Chain VL
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 17 aa, Artificial Sequence
  Synthetic Polypeptide
  MISC_FEATURE (5)..(5): Xaa is D or E
  MISC_FEATURE (6)..(6): Xaa is G or A
VIWYXXSNKHYADSVKG

SEQ ID NO: 14 - protein, 17 aa, Artificial Sequence
  Synthetic Polypeptide
VIWYEGSNKHYADSVKG

SEQ ID NO: 15 - protein, 17 aa, Artificial
  Synthetic Polypeptide
VIWYDASNKHYADSVKG

SEQ ID NO: 16 - protein, 17 aa, Artificial
  Synthetic Polypeptide
VIWYEASNKHYADSVKG

SEQ ID NO: 17 - protein, 7 aa, Artificial
  Synthetic Polypeptide
  MISC_FEATURE (1)..(1): Xaa is D or E
  MISC_FEATURE (2)..(2): Xaa is D or E
  MISC_FEATURE (3)..(3): Xaa is S or A
XXXDRPS

SEQ ID NO: 18 - protein, 7 aa, Artificial Sequence
  Synthetic Polypeptide
EDSDRPS

SEQ ID NO: 19 - protein, 7 aa, Artificial Sequence
  Synthetic Polypeptide
DESDRPS

SEQ ID NO: 20 - protein, 7 aa, Artificial Sequence
  Synthetic Polypeptide
EESDRPS

SEQ ID NO: 21 - protein, 7 aa, Artificial Sequence
  Synthetic Polypeptide
DDADRPS

SEQ ID NO: 22 - protein, 7 aa, Artificial Sequence
  Synthetic Polypeptide
DEADRPS

SEQ ID NO: 23 - protein, 7 aa, Artificial Sequence
  Synthetic Polypeptide
EDADRPS

SEQ ID NO: 24 - protein, 7 aa, Artificial Sequence
  Synthetic Polypeptide
EEADRPS

SEQ ID NO: 25 - protein, 122 aa, Artificial Sequence
  Synthetic Polypeptide
  MISC_FEATURE (54)..(54): Xaa is D or E
  MISC_FEATURE (55)..(55): Xaa is G or A
QMQLVESGGGVVQPGRSLRLSCAASGFTFRTYGMHWVRQAPGKGLEWVAVIWYXXSNKHYADSVKGRFTITRDNSKNTLNLQMNSLRAEDTAVYYCARAPQWELVHEAFDIWGQGTMVTVSS

SEQ ID NO: 26 - protein, 122 aa, Artificial Sequence
  Synthetic Polypeptide
QMQLVESGGGVVQPGRSLRLSCAASGFTFRTYGMHWVRQAPGKGLEWVAVIWYEGSNKHYADSVKGRFTITRDNSKNTLNLQMNSLRAEDTAVYYCARAPQWELVHEAFDIWGQGTMVTVSS

SEQ ID NO: 27 - protein, 122 aa, Artificial Sequence
  Synthetic Polypeptide
QMQLVESGGGVVQPGRSLRLSCAASGFTFRTYGMHWVRQAPGKGLEWVAVIWYDASNKHYADSVKGRFTITRDNSKNTLNLQMNSLRAEDTAVYYCARAPQWELVHEAFDIWGQGTMVTVSS

SEQ ID NO: 28 - protein, 122 aa, Artificial Sequence
  Synthetic Polypeptide
QMQLVESGGGVVQPGRSLRLSCAASGFTFRTYGMHWVRQAPGKGLEWVAVIWYEASNKHYADSVKGRFTITRDNSKNTLNLQMNSLRAEDTAVYYCARAPQWELVHEAFDIWGQGTMVTVSS

SEQ ID NO: 29 - protein, 108 aa, Artificial Sequence
  Synthetic Polypeptide
  MISC_FEATURE (49)..(49): Xaa is D or E
  MISC_FEATURE (50)..(50): Xaa is D or E
  MISC_FEATURE (51)..(51): Xaa is S or A
SYVLTQPPSVSVAPGQTARITCGGNNLGSKSVHWYQQKPGQAPVLVVYXXXDRPSWIPERFSGSNSGNTATLTISRGEAGDEADYYCQVWDSSSDHVVFGGGTKLTVL

SEQ ID NO: 30 - protein, 108 aa, Artificial Sequence
  Synthetic Polypeptide
SYVLTQPPSVSVAPGQTARITCGGNNLGSKSVHWYQQKPGQAPVLVVYEDSDRPSWIPERFSGSNSGNTATLTISRGEAGDEADYYCQVWDSSSDHVVFGGGTKLTVL

SEQ ID NO: 31 - protein, 108 aa, Artificial Sequence
  Synthetic Polypeptide
SYVLTQPPSVSVAPGQTARITCGGNNLGSKSVHWYQQKPGQAPVLVVYDESDRPSWIPERFSGSNSGNTATLTISRGEAGDEADYYCQVWDSSSDHVVFGGGTKLTVL

SEQ ID NO: 32 - protein, 108 aa, Artificial Sequence
  Synthetic Polypeptide
SYVLTQPPSVSVAPGQTARITCGGNNLGSKSVHWYQQKPGQAPVLVVYEESDRPSWIPERFSGSNSGNTATLTISRGEAGDEADYYCQVWDSSSDHVVFGGGTKLTVL

SEQ ID NO: 33 - protein, 108 aa, Artificial Sequence
  Synthetic Polypeptide
SYVLTQPPSVSVAPGQTARITCGGNNLGSKSVHWYQQKPGQAPVLVVYDDADRPSWIPERFSGSNSGNTATLTISRGEAGDEADYYCQVWDSSSDHVVFGGGTKLTVL

SEQ ID NO: 34 - protein, 108 aa, Artificial Sequence
  Synthetic Polypeptide
SYVLTQPPSVSVAPGQTARITCGGNNLGSKSVHWYQQKPGQAPVLVVYDEADRPSWIPERFSGSNSGNTATLTISRGEAGDEADYYCQVWDSSSDHVVFGGGTKLTVL

SEQ ID NO: 35 - protein, 108 aa, Artificial Sequence
  Synthetic Polypeptide
SYVLTQPPSVSVAPGQTARITCGGNNLGSKSVHWYQQKPGQAPVLVVYEDADRPSWIPERFSGSNSGNTATLTISRGEAGDEADYYCQVWDSSSDHVVFGGGTKLTVL

SEQ ID NO: 36 - protein, 108 aa, Artificial Sequence
  Synthetic Polypeptide
SYVLTQPPSVSVAPGQTARITCGGNNLGSKSVHWYQQKPGQAPVLVVYEEADRPSWIPERFSGSNSGNTATLTISRGEAGDEADYYCQVWDSSSDHVVFGGGTKLTVL

SEQ ID NO: 37 - protein, 448 aa, Artificial Sequence
  Synthetic Polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 38 - protein, 214 aa, Artificial Sequence
  Synthetic Polypeptide
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 140 aa, Artificial Sequence
  Synthetic Polypeptide
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRTTNKCLEQVSQLQGLWRRFNRPLLKQQHHHHHHDYKDDDDK

(19) United States
(12) Patent Application Publication
(10) Pub. No.: US 2024/0182558 A1
(43) Pub. Date: Jun. 6, 2024

(54) MODIFIED ANTI-TSLP ANTIBODIES

(71) Applicant: AMGEN INC., Thousand Oaks, CA (US)

(72) Inventors: Pavel Bondarenko, Thousand Oaks, CA (US); Liuqing Shi, San Mateo, CA (US); Hao Zhang, Lexington, MA (US)

(21) Appl. No.: 18/287,597

(22) PCT Filed: Apr. 22, 2022
(86) PCT No.: PCT/US22/25999

§ 371 Date: Oct. 19, 2023

Related U.S. Application Data
(60) Provisional application No. 63/178,915, filed on Apr. 23, 2021.

(51) Int. Cl. C07K 16/24; A61K 39/00; C12N 15/63

(52) U.S. Cl. CPC C07K 16/246; C12N 15/63; A61K 2039/505; A61K 2039/545; C07K 2317/14; C07K 2317/21; C07K 2317/565; C07K 2317/92; C07K 2317/94

(57) ABSTRACT

The present application relates, in general, to compositions or variants of anti-TSLP antibody tezepelumab having increased stability compared to tezepelumab when stored over long periods of time.

### CROSS-REFERENCE TO RELATED APPLICATIONS

The present application claims the priority benefit of U.S. Provisional Patent Application No. 63/178,915, filed Apr. 23, 2021, hereby incorporated by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55581_Seqlisting.txt”, which was created on Apr. 12, 2022 and is 32,649 bytes in size. The subject matter of the Sequence Listing is incorporated herein in its entirety by reference.

### FIELD OF THE DISCLOSURE

The present application relates, in general, to compositions and variants of anti-TSLP antibody tezepelumab having increased stability compared to tezepelumab when stored over long periods of time.

### BACKGROUND OF THE DISCLOSURE

Thymic stromal lymphopoietin (TSLP), an epithelial cell-derived cytokine produced in response to environmental and pro-inflammatory stimuli, leads to the activation of multiple inflammatory cells and downstream pathways (Soumelis et al. Nat Immunol 2002; 3:673-80; Allakhverdi et al. J Exp Med 2007; 204:253-8). TSLP is increased in the airways of patients with asthma and correlates with Th2 cytokine and chemokine expression (Shikotra et al. J Allergy Clin Immunol 2012; 129:104-11 e1-9) and disease severity (Ying et al. J Immunol 2005; 174:8183-90; Ying et al. J Immunol 2008; 181:2790-8). While TSLP is central to the regulation of Th2 immunity, it may also play a key role in other pathways of inflammation and therefore be relevant to multiple asthma phenotypes.

Tezepelumab is a human immunoglobulin G2 (IgG2) monoclonal antibody (mAb) that binds to TSLP, preventing its interaction with the TSLP receptor complex. A proof-of-concept study in patients with mild, atopic asthma, demonstrated that tezepelumab inhibited the early and late asthmatic responses and suppressed biomarkers of Th2 inflammation following inhaled allergen challenge (Gauvreau, et al. N Engl J Med 2014; 370:2102-10).

### SUMMARY

Monitoring of antibody therapeutics in formulation over time is important to determine storage conditions that reduce any breakdown of the therapeutic and maintain the integrity of the product. The present disclosure provides a study of attributes of an anti-TSLP antibody that can change over time in storage and attributes that can be beneficial or detrimental to antibody stability.

In one aspect, the disclosure provides an anti-TSLP immunoglobulin, antigen binding protein or fragment thereof, or antibody or fragment thereof comprising (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising an amino acid sequence with a mutation at at least one of the following residues, D54 or G55 set forth in SEQ ID NO:7, and (iii) a heavy chain CDR3 sequence comprising the amino acid sequence set forth in SEQ ID NO:8. In various embodiments, the HCDR2 has the sequence VIWYX₁X₂SNKHYADSVKG (SEQ ID NO: 13), wherein X₁ is D or E and X₂ is G or A. In various embodiments, the HCDR2 has the following sequence: VIWYEGSNKHYADSVKG (SEQ ID NO: 14), VIWYDASNKHYADSVKG (SEQ ID NO: 15) or VIWYEASNKHYADSVKG (SEQ ID NO: 16).

In various embodiments, the mutation in HCDR2 is D54E. In various embodiments, the mutation in HCDR2 is G55A. In various embodiments, the anti-TSLP antigen binding protein or fragment thereof optionally comprises a mutation in at least one of the following residues of LCDR2 D49, D50, or S51 of SEQ ID NO: 4. In various embodiments, the mutation of LCDR2 is one or more of D49E, D50E, or S51A. In various embodiments, the LCDR2 has the sequence X₁X₂X₃DRPS, wherein X₁ is D or E, X₂ is D or E, and X₃ is S or A (SEQ ID NO: 17). In various embodiments, the LCDR2 has the following sequence: EDSDRPS (SEQ ID NO: 18), DESDRPS (SEQ ID NO: 19), EESDRPS (SEQ ID NO: 20), DDADRPS (SEQ ID NO: 21), DEADRPS (SEQ ID NO: 22), EDADRPS (SEQ ID NO: 23) or EEADRPS (SEQ ID NO: 24).

In various embodiments, the disclosure provides an anti-TSLP immunoglobulin, antigen binding protein or fragment thereof, or antibody or fragment thereof comprising (A) a light chain variable domain comprising: (i) a light chain CDR1 sequence comprising the amino acid sequence set forth in SEQ ID NO: 3; (ii) a light chain CDR2 sequence comprising an amino acid sequence with a mutation in at least one of the following residues D49, D50, or S51 of SEQ ID NO: 4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO: 6; (ii) a heavy chain CDR2 sequence comprising the amino acid sequence set forth in SEQ ID NO: 7 and (iii) a heavy chain CDR3 sequence comprising the amino acid sequence set forth in SEQ ID NO: 8.

In various embodiments, the LCDR2 has the sequence X₁X₂X₃DRPS, wherein X₁ is D or E, X₂ is D or E, and X₃ is S or A (SEQ ID NO: 17). Optionally the LCDR2 has the following sequence: EDSDRPS (SEQ ID NO: 18), DESDRPS (SEQ ID NO: 19), EESDRPS (SEQ ID NO: 20), DDADRPS (SEQ ID NO: 21), DEADRPS (SEQ ID NO: 22), EDADRPS (SEQ ID NO: 23) or EEADRPS (SEQ ID NO: 24). In various embodiments, the mutation in LCDR2 is D49E. In various embodiments, the mutation in LCDR2 is D50E. In various embodiments, the mutation in LCDR2 is S51A. In various embodiments, the anti-TSLP immunoglobulin, antigen binding protein or fragment thereof, or antibody or fragment thereof optionally comprises a mutation at one of the following residues D54 or G55 in HCDR2 set out in SEQ ID NO: 7. In various embodiments, the mutation in HCDR2 is one or more of D54E or G55A in SEQ ID NO: 7. In various embodiments, the HCDR2 has the sequence VIWYX₁X₂SNKHYADSVKG (SEQ ID NO: 13), wherein X₁ is D or E and X₂ is G or A. In various embodiments, the HCDR2 has the following sequence: VIWYEGSNKHYADSVKG (SEQ ID NO: 14), VIWYDASNKHYADSVKG (SEQ ID NO: 15) or VIWYEASNKHYADSVKG (SEQ ID NO: 16).

In various embodiments, the disclosure provides an anti-TSLP immunoglobulin, antigen binding protein or fragment thereof, or antibody or fragment thereof comprising (A) a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; or iii. a sequence of amino acids encoded by a polynucleotide that hybridizes under moderately stringent conditions to the complement of a polynucleotide consisting of SEQ ID NO:11; or (B) a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; or iii. a sequence of amino acids encoded by a polynucleotide that hybridizes under moderately stringent conditions to the complement of a polynucleotide consisting of SEQ ID NO:9; or (C) a light chain variable domain of (A) and a heavy chain variable domain of (B), wherein the anti-TSLP immunoglobulin, antigen binding protein or fragment thereof, or antibody or fragment thereof retains one or more of CDRs of the anti-TSLP antigen binding proteins or fragment thereof and comprises a mutation at one or more of HCDR2 D54 or G55 of SEQ ID NO: 7, or LCDR2 D49, D50, or S51 of SEQ ID NO: 4.

In various embodiments, the anti-TSLP immunoglobulin, antigen binding protein or fragment thereof, or antibody or fragment thereof comprises a heavy chain comprising the amino acid sequence of SEQ ID NO: 10 or SEQ ID NO: 25-28, and a light chain comprising the amino acid sequence of SEQ ID NO: 12 or SEQ ID NO: 29-36.

In various embodiments, the anti-TSLP immunoglobulin, antigen binding protein or fragment thereof, or antibody or fragment thereof comprises an anti-TSLP antigen binding protein selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgM antibody, an IgG1 antibody, an IgG2 antibody, an IgG3 antibody, and an IgG4 antibody.

In various embodiments, the immunoglobulin, antigen binding protein or antibody is a human antibody. In various embodiments, the antibody is an IgG2 antibody. In various embodiments, the anti-TSLP immunoglobulin, antigen binding protein or fragment thereof, or antibody or fragment thereof specifically binds to a TSLP polypeptide as set forth in amino acids 29-159 of SEQ ID NO: 2. In various embodiments, both binding sites of the anti-TSLP immunoglobulin, antigen binding protein or fragment thereof, or antibody or fragment thereof have identical binding to TSLP.

In various embodiments, the anti-TSLP immunoglobulin, antigen binding protein or fragment thereof, or antibody or fragment thereof binds TSLP with an affinity of that is numerically no more than 10⁻⁸ M Kd.

Further contemplated is a composition comprising the anti-TSLP immunoglobulin, antigen binding protein or fragment thereof, or antibody or fragment thereof of described herein and a pharmaceutically acceptable carrier, excipient or diluent.

The disclosure also provides an isolated nucleic acid comprising a polynucleotide sequence encoding the light chain variable domain, the heavy chain variable domain, or both, of the immunoglobulin, antigen binding protein or antibody described herein.

The disclosure further contemplates a recombinant expression vector comprising the nucleic acid encoding an anti-TSLP immunoglobulin, antigen binding protein or antibody as described herein. Also provided is a host cell comprising the expression vector.

Further contemplated herein is a method of producing an immunoglobulin, antigen binding protein or fragment thereof, or antibody or fragment thereof that specifically binds to a TSLP polypeptide comprising amino acids 29-159 of SEQ ID NO: 2, comprising incubating the host cell under conditions that allow it to express the immunoglobulin, antigen binding protein, or antibody, wherein said host cell comprises (i) a recombinant expression vector encoding the light chain variable domain of the antigen binding protein of as described herein and a recombinant expression vector encoding the heavy chain variable domain of the antigen binding protein as described herein, or (ii) a recombinant expression vector encoding both the light chain variable domain and the heavy chain variable domain of the immunoglobulin, antigen binding protein or antibody as described herein.

In various embodiments, the anti-TSLP immunoglobulin, antigen binding protein or fragment thereof, or antibody or fragment thereof has increased stability at 25° C. compared to an anti-TSLP immunoglobulin, antigen binding protein or fragment thereof, or antibody or fragment thereof having the amino acid sequences set out in SEQ ID NO: 10 and SEQ ID NO: 12. In various embodiments, the anti-TSLP immunoglobulin, antigen binding protein or fragment thereof, or antibody or fragment thereof has increased stability at 40° C. after 4 weeks compared to an anti-TSLP immunoglobulin, antigen binding protein or fragment thereof, or antibody or fragment thereof having the amino acid sequences set out in SEQ ID NO: 10 and SEQ ID NO: 12. In various embodiments, the anti-TSLP immunoglobulin, antigen binding protein or fragment thereof, or antibody or fragment thereof has decreased high molecular weight species at 40° C. after 4 weeks compared to an anti-TSLP antigen binding protein or fragment thereof having the amino acid sequences set out in SEQ ID NO: 10 and SEQ ID NO: 12.

In various embodiments, the anti-TSLP immunoglobulin, antigen binding protein or fragment thereof, or antibody or fragment thereof had decreased isomerization at 50° C. compared to an anti-TSLP immunoglobulin, antigen binding protein or fragment thereof, or antibody or fragment thereof having the amino acid sequences set out in SEQ ID NO: 10 and SEQ ID NO: 12.

In various embodiments, less than 2% of the anti-TSLP immunoglobulin, antigen binding protein or fragment thereof, or antibody or fragment thereof shows isomerization after at least 2 weeks (optionally, after at least 1 month, after at least 2 months, after at least 3 months, after at least 4 months, after at least 5 months or after at least 6 months) of storage at about 25° C., as determined by SEC, e.g. SEC of antibody-antigen complex. In various embodiments, less than 2% of the antigen binding protein or fragment thereof shows isomerization after about 22 months to about 36 months of storage at 2° C. to 8° C. followed by at least 2 weeks or at least 1 month or at least 2 months or at least 3 months of storage at about 25° C., as determined by SEC.

Also provided herein is a method for treating an inflammatory disease in a subject comprising administering to the subject a therapeutically effective amount of the immunoglobulin, antigen binding protein or fragment thereof, or antibody or fragment thereof as described herein or a composition thereof. In various embodiments, the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF). In various embodiments, the asthma is mild, moderate or severe asthma. In various embodiments, the asthma is severe asthma. In various embodiments, the asthma is eosinophilic or non-eosinophilic asthma.

In various embodiments, the method comprises administering the composition at an interval of every 2 weeks or every 4 weeks. In various embodiments, the composition is administered for a period of at least 4 months, 6 months, 9 months, 1 year or more.

In various embodiments, the disclosure provides a method of making a composition comprising a plurality of anti-TSLP monoclonal antibodies or antigen binding fragments thereof each comprising: a light chain CDR1 sequence comprising the amino acid sequence set forth in SEQ ID NO:3; a light chain CDR2 sequence comprising the amino acid sequence set forth in SEQ ID NO:4; a light chain CDR3 sequence comprising the amino acid sequence set forth in SEQ ID NO:5; a heavy chain CDR1 sequence comprising the amino acid sequence set forth in SEQ ID NO:6; a heavy chain CDR2 sequence comprising the amino acid sequence set forth in SEQ ID NO: 7; and a heavy chain CDR3 sequence comprising the amino acid sequence set forth in SEQ ID NO:8, the method comprising enriching the composition for IgG2 anti-TSLP monoclonal antibodies that comprise at least one of: L-aspartate at HC position 54, relative to isoAspartate (isoAsp) or cyclic aspartate (cAsp) in HCDR2 set out in SEQ ID NO: 7; non-oxidized HC W102, relative to oxidized W102 in HCDR3 set out in SEQ ID NO: 8; L-aspartate at LC position 49 or position 50, relative to isoAsp or cAsp in LCDR2 set out in SEQ ID NO: 4; LC N65 relative to deamidated N65 set out in LC SEQ ID NO: 12; or L-aspartate at LC position 91 of LCDR3 set out in SEQ ID NO: 5, relative to isoAsp or cAsp. cAsp is also known as succinimide featuring H₂O loss relative to Asp or isoAsp.

In various embodiments, less than 2.0% of the anti-TSLP monoclonal antibodies comprise isomerized HC D54. In various embodiments, no more than 0.9% of the anti-TSLP monoclonal antibodies comprise isomerized HC D54. In various embodiments, no more than 2% of the anti-TSLP monoclonal antibodies comprise oxidized HC W102. In various embodiments, less than 2.0% of the anti-TSLP monoclonal antibodies comprise isomerized LC D49 or D50. In various embodiments, no more than 0.9% of the anti-TSLP monoclonal antibodies comprise isomerized LC D49 or D50. In various embodiments, no more than 0.5% of the anti-TSLP monoclonal antibodies comprise deamidated LC N65. In various embodiments, no more than 0.9% of the anti-TSLP monoclonal antibodies comprise isomerized LC D91.

Further contemplated is a composition comprising anti-TSLP monoclonal antibodies each comprising: a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ ID NO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO: 8, the composition comprising a limited content of isomerized HC D54 (SEQ ID NO: 7) and/or a limited content of isomerized LC D49 or D50 (SEQ ID NO: 4), effective for the anti-TSLP monoclonal antibodies of the composition to bind to TLSP with a Kd that is numerically less than or equal to 10⁻⁸ M.

Also provided is a composition comprising IgG2 anti-TSLP monoclonal antibodies, each comprising a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ ID NO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO: 8, wherein at least one of: no more than 0.9% of the anti-TSLP monoclonal antibodies comprise isomerized HC D54; no more than 2% of the anti-TSLP monoclonal antibodies comprise oxidized HC W102; no more than 6.7% of the anti-TSLP monoclonal antibodies comprise isomerized LC D49 or D50; no more than 0.5% of the anti-TSLP monoclonal antibodies comprise deamidated LC N65; or no more than 0.9% of the anti-TSLP monoclonal antibodies comprise isomerized LC D91. In various embodiments, no more than 0.9% of the anti-TSLP monoclonal antibodies comprise isomerized HC D54. In various embodiments, no more than 2% of the anti-TSLP monoclonal antibodies comprise oxidized HC W102. In various embodiments, no more than 0.9% of the anti-TSLP monoclonal antibodies comprise isomerized LCD49 or LC D50. In various embodiments, no more than 0.5% of the anti-TSLP monoclonal antibodies comprise deamidated LC N65. In various embodiments, no more than 0.9% of the anti-TSLP monoclonal antibodies comprise isomerized LC D91.

Also provided is a composition comprising IgG2 anti-TSLP monoclonal antibodies, each comprising a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ ID NO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO:8, wherein at least one of: no more than 0.9% of the anti-TSLP monoclonal antibodies comprise isomerized HC D54; no more than 2% of the anti-TSLP monoclonal antibodies comprise oxidized HC W102; no more than 12.9% of the anti-TSLP monoclonal antibodies comprise isomerized LC D49 or D50; no more than 0.5% of the anti-TSLP monoclonal antibodies comprise deamidated LC N65; or no more than 0.9% of the anti-TSLP monoclonal antibodies comprise isomerized LC D91. In various embodiments, no more than 0.9% of the anti-TSLP monoclonal antibodies comprise isomerized HC D54. In various embodiments, no more than 2% of the anti-TSLP monoclonal antibodies comprise oxidized HC W102. In various embodiments, no more than 0.9% of the anti-TSLP monoclonal antibodies comprise isomerized LCD49 or LC D50. In various embodiments, no more than 0.5% of the anti-TSLP monoclonal antibodies comprise deamidated LC N65. In various embodiments, no more than 0.9% of the anti-TSLP monoclonal antibodies comprise isomerized LC D91.

In various embodiments, the anti-TSLP antibody comprises a combination of L-aspartate at HC D54 and L-aspartate at LC D49 or D50. In various embodiments, the anti-TSLP antibody is enriched in L-aspartate at HC D54 to at least 6-fold over the levels of isoAsp.

In various embodiments, the antibody is an IgG2 antibody. In various embodiments, the anti-TSLP antibody comprises a heavy chain variable region set out in SEQ ID NO: 10 or SEQ ID NOs: 25-28 and a light chain variable region set out in SEQ ID NO: 12 or SEQ ID NOS: 29-36, and comprises one or more of the sequence modifications described herein.

The disclosure also provides a composition comprising anti-TSLP monoclonal antibodies, each comprising a light chain CDR1 sequence comprising the amino acid sequence set forth in SEQ ID NO:3; a light chain CDR2 sequence comprising the amino acid sequence set forth in SEQ ID NO:4; a light chain CDR3 sequence comprising the amino acid sequence set forth in SEQ ID NO:5; a heavy chain CDR1 sequence comprising the amino acid sequence set forth in SEQ ID NO:6; a heavy chain CDR2 sequence comprising the amino acid sequence set forth in SEQ ID NO: 7; and a heavy chain CDR3 sequence comprising the amino acid sequence set forth in SEQ ID NO:8, wherein at least one of: greater than 98% of the anti-TSLP monoclonal antibodies of the composition comprise L-aspartate at HC position 54, relative to isoAsp or cAsp at position 54 (SEQ ID NO: 7); at least 99% of the anti-TSLP monoclonal antibodies of the composition comprise non-oxidized HC W102 relative to oxidized W102 (SEQ ID NO: 8); at least 97% of the anti-TSLP monoclonal antibodies of the composition comprise L-aspartate at LC position 49 or position 50, relative to isoAsp or cAsp at position 49 or 50 (SEQ ID NO: 4); at least 99.1% of the anti-TSLP monoclonal antibodies of the composition comprise LC N65 relative to deamidated LC N65 (SEQ ID NO: 12); or at least 99.1% of the anti-TSLP monoclonal antibodies of the composition comprise L-aspartate at LC position 91, relative to isoAsp or cAsp at position 91 (SEQ ID NO: 5).

The disclosure also provides a composition comprising an anti-TSLP antibody or antigen binding fragment thereof as described herein for use in treating an inflammatory disease as described herein. In certain embodiments, the disclosure provides use of a composition comprising an anti-TSLP antibody or antigen binding fragment thereof as described herein in the preparation of a medicament for treating an inflammatory disease.

Syringes, e.g., single use or pre-filled syringes, sterile sealed containers, e.g. vials, bottle, vessel, and/or kits or packages comprising any of the foregoing antibodies or compositions, optionally with suitable instructions for use, are also contemplated.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawings and/or claims.

### BRIEF DESCRIPTION OF THE DRAWINGS

FIG. 1 illustrates the workflow for characterizing residues and modifications that potentially impact binding determined by the SEC affinity binding of heat stressed AMG157 (40C4W) and TSLP. In silico sequence analysis is shown (top). Several mutations of residues with modifications were considered to improve the stability of AMG 157 at room temperature (bottom).

FIG. 2 shows relative abundance of potential attributes in the T0, 40C4W and 22M5C+2M25C samples of AMG 157 as determined by SEC of antigen-antibody complex. These attributes were predicted as potentially impacting TSLP stability according to in silico sequence (as shown in FIG. 1). White, black and gray bars represent the modification percentages in AMG 157 T0, 40C4W and 22M5C+2M25C samples, respectively. A dashed line is shown to represent 2%.

FIG. 3A shows SEC-UV profiles of AMG157 T0, AMG157 40C4W, TSLP, AMG157 T0+TSLP mixture, and AMG157 40C4W+TSLP mixture. Five SEC-UV peak regions are assigned based on the peak shape and molecular weight. Peak 3 represents the bound fraction of AMG 157 with TSLP, and peak 5 corresponds to the unbound fraction of AMG 157. The cartoon of each assigned peak is shown on top of the corresponding peak. FIG. 3B shows modification percentages of five attributes in the bound and unbound fractions of the SEC binding.

FIG. 4 is a volcano plot showing how the attributes of AMG 157 are distributed in statistics for impacting the TSLP binding. Attributes appearing in the top right corner are the modifications of AMG 157 that potentially impact TSLP binding. X-axis is log 2 value of the change fold between unbound and bound, and y-axis is −log 10 value of the p-value that represents the statistical significance. The gray area is considered as the background.

FIGS. 5A and 5B show modification percentages of the bound and unbound AMG157 fractions in 10 residues that were considered as potentially important based on in silico sequence analysis. However, SEC of antibody-antigen revealed that he ratio of the modifications in unbound versus bound fractions is not statistically different. It is hypothesized that the modifications do not impact binding as measured by the SEC of antibody-antigen method. FIGS. 5A and 5B have the percentage scale of 0-50% and 0-1%, respectively.

FIG. 6 shows SEC-UV profiles of AMG 157 T0, 40° C.4W, and 50° C.1W. The SEC-UV profiles of AMG 157 T0, 40° C.4W, and 50° C.1W are shown in black solid line, blue dotted line, and red dashed line, respectively. Based on the elution time and theoretical molecular weight of AMG 157, the peak eluting at ˜10.5 min is assigned as high molecular weight species of AMG 157 (HMW), and the peak eluting at ˜15.5 min is assigned as the monomer of AMG 157. According to the integrated peak areas, the percentage of HMW species in 40° C.4W and 50° C.1W are ˜9% and 67%, respectively, and are indicated in the top left of the figure.

FIG. 7A shows a volcano plot showing how the attributes of AMG 157 after 50C1W stress are distributed between HMW and monomer species and statistical significance. Attributes appearing in the top right corner are modifications of AMG 157 that correlate to formation of HMW in 50C1W. X-axis is log 2 value of the fold change between HMW and monomer species, and y-axis is −log 10 value of the p-value that represents the statistical significance. The gray area is considered as the background noise, but also may contain true values with lower confidence. FIG. 7B shows percentages of 20 modifications in the HMW species and monomer of AMG 157 50C1W sample. These 20 modifications including statistically significant modifications from the top right white corner (7 modifications market with asterisks) and modifications from adjacent “gray area with near statistical significance”. Each of these 20 modifications has relatively high values of fold change and significance. Sum of the −log 10 of p-value and log 2 value of fold change for these 20 modifications was >4.6.

FIG. 8A shows isomerization levels measurements by peptide mapping and potency measurements of the antibody drug substance and antibody stressed for 4 weeks at 40 C. FIG. 8B shows CEX-HPLC profile of the antibody. FIG. 8C shows results of the traditional method of characterization of CEX fractions of AMG 157 40° C.4W sample for chemical modifications by peptide mapping and for relative potency. FIG. 8D shows results of long-term stability studies, approaching end of shelf life.

### DETAILED DESCRIPTION

It is further contemplated that treatment with tezepelumab could eliminate daily disease activity and make more patients steroid-free or reduce the need for use of steroids in the treatment of inflammatory diseases, such as asthma.

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “inflammatory disease” refers to a medical condition involving abnormal inflammation caused by the immune system attacking the body's own cells or tissues, which may result in chronic pain, redness, swelling, stiffness, and damage to normal tissues. Inflammatory diseases include, for example, asthma, chronic peptic ulcer, tuberculosis, periodontitis, sinusitis, active hepatitis, ankylosing spondylitis, rheumatoid arthritis, chronic obstructive pulmonary disease (COPD), Crohn's disease, ulcerative colitis, osteoarthritis, atherosclerosis, systemic lupus erythematosus, atopic dermatitis, eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps, idiopathic pulmonary fibrosis (IPF), and the like. In exemplary aspects, the inflammatory disease is asthma, atopic dermatitis, or COPD. In exemplary aspects, the inflammatory is asthma and, in some instances, the asthma is severe asthma, eosinophilic asthma, non-eosinophilic asthma, or low eosinophil asthma.

The term “asthma” as used herein refers to allergic, non-allergic, eosinophilic, and non-eosinophillic asthma.

The term “allergic asthma” as used herein refers to asthma that is triggered by one or more inhaled allergens. Such patients have a positive IgE fluorescence enzyme immunoassay (FEIA) level to one or more allergens that trigger an asthmatic response. Typically, most allergic asthma is associated with Th2-type inflammation.

The term “non-allergic asthma” refers to patients that have low eosinophil, low Th2, or low IgE at the time of diagnosis. A patient who has “non-allergic asthma” is typically negative in the IgE fluorescence enzyme immunoassay (FEIA) in response to a panel of allergens, including region-specific allergens. In addition to low IgE, those patients often have low or no eosinophil counts and low Th2 counts at the time of diagnosis.

The term “severe asthma” as used herein refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

The term “eosinophilic asthma” as used herein refers to an asthma patient having a screening blood eosinophil count of less than or equal to 300 cells/μL, or less than or equal to 250 cells/μL “Low eosinophilic” asthma refers to asthma patients having less than 250 cells/μL blood or serum. Alternatively, “low eosinophilic” asthma refers to asthma patients having less than 300 cells/μL blood or serum.

A “T helper (Th) 1 cytokine” or “Th1-specific cytokine” refers to cytokines that are expressed (intracellularly and/or secreted) by Th1 T cells, and include IFN-g, TNF-a, and IL-12. A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-a, IL-2 and IL-17.

The term “specifically binds” is “antigen specific”, is “specific for”, “selective binding agent”, “specific binding agent”, “antigen target” or is “immunoreactive” with an antigen refers to an antibody or polypeptide that binds an target antigen with greater affinity than other antigens of similar sequence. It is contemplated herein that the agent specifically binds target proteins useful in identifying immune cell types, for example, a surface antigen (e.g., T cell receptor, CD3), a cytokine (e.g., TSLP, IL-4, IL-5, IL-13, IL-17, IFN-g, TNF-a) and the like. In various embodiments, the antibody specifically binds the target antigen, but can cross-react with an ortholog of a closely related species, e.g. an antibody may being human protein and also bind a closely related primate protein. In various embodiments, the immunoglobulin, antigen binding protein or fragment thereof, or antibody or fragment thereof specific for TLSP binds with a Kd that is numerically less than or equal to 10⁻⁸ M. In various embodiments, an anti-TSLP antibody described herein binds at least with an affinity (Kd) of 10⁻⁸ M, 10⁻⁹ M, 10⁻¹⁰ M, 10⁻¹¹ M, 10⁻¹² M, 10⁻¹³ M or less.

The term “antibody” refers to a tetrameric glycoprotein that consists of two heavy chains and two light chains, each comprising a variable region and a constant region. “Heavy Chains” and “Light Chains” refer to substantially full length canonical immunoglobulin light and heavy chains (see e.g., Immunobiology, 5th Edition (Janeway and Travers et al., Eds., 2001). Antigen-binding portions may be produced by recombinant DNA techniques or by enzymatic or chemical cleavage of intact antibodies.

Antigen binding proteins include antibodies, antibody fragments and antibody-like proteins that can have structural changes to structure of canonical tetrameric antibodies. Antibody “variants” refer to antigen binding proteins or fragments thereof that can have structural changes in antibody sequence or function compared to a parent antibody having a known sequence. Antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody fragments include antigen-binding portions of the antibody including, inter alia, Fab, Fab′, F(ab′)2, Fv, domain antibody (dAb), complementarity determining region (CDR) fragments, CDR-grafted antibody binding regions, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Valency” refers to the number of antigen binding sites on each antibody or antibody fragment that targets an epitope. A typical full length IgG molecule, or F(ab)2 is “bivalent” in that it has two identical target binding sites. A “monovalent’ antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity” includes inhibiting any one or more of the following: binding of TSLP to its receptor; proliferation, activation, or differentiation of cells expressing TSLPR in the presence of TSLP; inhibition of Th2 cytokine production in a polarization assay in the presence of TSLP; dendritic cell activation or maturation in the presence of TSLP; and mast cell cytokine release in the presence of TSLP. See, e.g., U.S. Pat. No. 7,982,016 B2, column 6 and example 8 and US 2012/0020988 A1, examples 7-10.

The term “sample” or “biological sample” refers to a specimen obtained from a subject for use in the present methods, and includes urine, whole blood, plasma, serum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

### TSLP

Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+ progenitor cells (Swedin et al. Pharmacol Ther 2017; 169:13-34). TSLP signals through a heterodimeric receptor consisting of the interleukin (IL)-7 receptor alpha (IL-7Rα) chain and a common γ chain-like receptor (TSLPR) (Pandey, Nat Immunol. 1:59-64, 2000; Park, J Exp Med. 192:659-669, 2000).

Human TSLP mRNA (Brightling et al., J Allergy Clin Immunol 2008; 121:5-10; quiz 1-2; Ortega et al. N Engl J Med 2014; 371:1198-207) and protein levels (Ortega et al., supra) are increased in the airways of asthmatic individuals compared to controls, and the magnitude of this expression correlates with disease severity (Brightling et al., supra). Recent studies have demonstrated association of a single nucleotide polymorphism in the human TSLP locus with protection from asthma, atopic asthma and airway hyperresponsiveness, suggesting that differential regulation of TSLP gene expression might influence disease susceptibility (Ortega et al. N Engl J Med 2014; 371:1198-207; To et al. BMC Public Health 2012; 12:204). These data suggest that targeting TSLP may inhibit multiple biological pathways involved in asthma.

Earlier non-clinical studies of TSLP suggested that after TSLP is released from airway epithelial cells or stromal cells, it activates mast cells, dendritic cells, and T cells to release Th2 cytokines (e.g., IL-4/13/5). Recently published human data demonstrated a good correlation between tissue TSLP gene and protein expression, a Th2 gene signature score, and tissue eosinophils in severe asthma. Therefore, an anti-TSLP target therapy may be effective in asthmatic patients with Th2-type inflammation (Shikotra et al., J Allergy Clin Immunol. 129(1):104-11, 2012).

Data from other studies suggest that TSLP may promote airway inflammation through Th2 independent pathways such as the crosstalk between airway smooth muscle and mast cells (Allakhverdi et al., J Allergy Clin Immunol. 123(4):958-60, 2009; Shikotra et al., supra). TSLP can also promote induction of T cells to differentiate into Th-17-cytokine producing cells with a resultant increase in neutrophilic inflammation commonly seen in more severe asthma (Tanaka et al., Clin Exp Allergy. 39(1):89-100, 2009). These data and other emerging evidence suggest that blocking TSLP may serve to suppress multiple biologic pathways including but not limited to those involving Th2 cytokines (IL-4/13/5).

### Antibodies

It is contemplated that antibodies or antibody variants or antigen binding proteins specific for TSLP are useful in the treatment of asthma, including severe asthma, eosinophlic asthma, no-eosinophilic/low-eosinophilic and other forms of asthma described herein, atopic dermatitis, and COPD.

Specific binding agents such as antibodies and antibody variants or fragments that bind to their target antigen, e.g., TSLP, are useful in the methods of the invention. In one embodiment, the specific binding agent is an antibody. The antibodies may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also encompassed by the invention are human antibody variants (including antibody fragments) that bind TSLP. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

Anti-TSLP antibody tezepelumab is described in U.S. Pat. No. 7,982,016 and U.S. patent application Ser. No. 15/951,602. It was discovered herein that under stressed storage conditions, e.g. 40° C. for 4 weeks (40C4W) or 50° C. for one week (50C1W), residues on the tezepelumab antibody undergo changes such as isomerization, deamidation or oxidation, that are detrimental to antibody stability. Residues identified as sources of reduced stability in anti-TSLP antibody tezepelumab CDRs (SEQ ID NOs: 3-8) or in the variable region (SEQ ID NOS: 10 and 12) include CDRH1 M34, CDRH2 W52, CDRH2 D54, CDRH2 N57, CDRH2 D62, CDRH3 W102, FRH1 N25, FRH1 N26, CDRL2 D49, CDRL2 D50, FRL2 N65, CDRL3 W90, CDRL3 D91, CDRL3 S92,S93,S94, CDRL3 D95. Numbering of the residues in tezepelumab is based on the heavy chain and light chain sequences set out in SEQ ID NO: 10 and 12, respectively.

Anti-TSLP antigen binding protein (including fragments thereof) useful in the present methods comprise an anti-TSLP antibody comprising a. a light chain variable domain comprising: i. a light chain CDR1 sequence comprising the amino acid sequence set forth in SEQ ID NO: 3; ii. a light chain CDR2 sequence comprising the amino acid sequence set forth in SEQ ID NO: 4; iii. a light chain CDR3 sequence comprising the amino acid sequence set forth in SEQ ID NO: 5; and, b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO: 7, and iii. a heavy chain CDR3 sequence comprising the amino acid sequence set forth in SEQ ID NO: 8, wherein the antibody or antibody variant specifically binds to a TSLP polypeptide as set forth in amino acids 29-159 of SEQ ID NO:2.

Also contemplated is an antibody or antibody variant comprising a. a light chain variable domain selected from the group consisting of: i. a sequence of amino acids at least 80% identical to SEQ ID NO: 12; ii. a sequence of amino acids encoded by a polynucleotide sequence that is at least 80% identical to SEQ ID NO: 11; iii. a sequence of amino acids encoded by a polynucleotide that hybridizes under moderately stringent conditions to the complement of a polynucleotide consisting of SEQ ID NO: 11; and, b. a heavy chain variable domain selected from the group consisting of: i. a sequence of amino acids that is at least 80% identical to SEQ ID NO: 10; ii. a sequence of amino acids encoded by a polynucleotide sequence that is at least 80% identical to SEQ ID NO: 9; iii. a sequence of amino acids encoded by a polynucleotide that hybridizes under moderately stringent conditions to the complement of a polynucleotide consisting of SEQ ID NO: 9; or c. a light chain variable domain of (a) and a heavy chain variable domain of (b), wherein the antibody or antibody variant specifically binds to a TSLP polypeptide as set forth in amino acids 29-159 of SEQ ID NO:2.

Tezepelumab is an exemplary anti-TSLP antibody having: a. i. a light chain CDR1 sequence comprising the amino acid sequence set forth in SEQ ID NO: 3; ii. a light chain CDR2 sequence comprising the amino acid sequence set forth in SEQ ID NO: 4; iii. a light chain CDR3 sequence comprising the amino acid sequence set forth in SEQ ID NO: 5; and b. a heavy chain variable domain comprising: i. a heavy chain CDR1 sequence comprising the amino acid sequence set forth in SEQ ID NO: 6; ii. a heavy chain CDR2 sequence comprising the amino acid sequence set forth in SEQ ID NO: 7, and ili. a heavy chain CDR3 sequence comprising the amino acid sequence set forth in SEQ ID NO: 8.

Tezepelumab also comprises a light chain variable domain having the amino acid sequence set out in SEQ ID NO: 12; encoded by a polynucleotide sequence set out in SEQ ID NO: 11; and a heavy chain variable domain having the amino acid sequence set out in SEQ ID NO: 10, encoded by a polynucleotide sequence set out in SEQ ID NO: 9.

Tezepelumab is an IgG2 antibody. The sequence of the full length heavy chain and light chain of tezepelumab, including the IgG2 chain, is set out in SEQ ID NOs: 37 and 38, respectively.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

In various embodiments, the anti-TSLP antibody variant is selected from the group consisting of a diabody, a triabody, a tetrabody, a Fab fragment, single domain antibody, scFv, wherein the dose is adjusted such that the binding sites to be equimolar to the those dosed by bivalent antibodies.

It is contemplated that the antibody or antibody variant is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, or to increase or decrease the affinity of the antibodies to human TSLP described herein.

According to certain embodiments, preferred amino acid substitutions are those which: (1) reduce susceptibility to proteolysis, (2) reduce susceptibility to oxidation, (3) alter binding affinity affinities, (4) inhibit formation of high molecular weight (HMW) species, and/or (5) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

### Identification of Attributes Contributing to Stability and Protein Binding

In order to determine attributes that contribute to protein binding and activity, the anti-TSLP antigen binding protein described herein is placed in a condition that leads to a change in its structure, for example, a change in the structure of an amino acid of the therapeutic protein, leading to the formation of a species of the therapeutic protein. In exemplary aspects, the changed structure of an amino acid is referred to as an “attribute” and may be characterized in terms of its chemical identity or attribute type and location within the amino acid sequence of the antigen binding protein, e.g., the position of the amino acid on which the attribute is present. For example, asparagine and glutamine residues are susceptible to deamidation. A deamidated asparagine at position 10 of a protein amino acid sequence is an example of an attribute. A list of exemplary attribute types for particular amino acids is provided in Table A. As such, a “structure” as used herein can comprise, consist essentially of, or consisting of an attribute type listed in Table A, or a combination of two or more attribute types listed in Table A. It will be understood that attributes are examples of structures, and unless stated otherwise, wherever a “structure” is mentioned herein, an attribute is contemplated as an example of the structure. For example, high molecular weight species (HMW) and fragments are also examples of attributes.

TABLE A

| Exemplary Attribute Type | Amino acid residue |
|---|---|
| deamidation | Asn, Gln |
| deamination | Glu, Ser, Gly |
| glycation, hydroxylysine | Lys |
| glycosylation | Asn |
| cyclization | N-terminal Gln, N-terminal Glu |
| oxidation | Met, Trp, His |
| isomerization | Asp |
| fragmentation/clipping | Asp/Pro |

As an immunoglobulin or fragment thereof, antibody or antigen binding protein comprises multiple amino acids, an antibody or antigen binding protein described herein may have more than one attribute (e.g., more than one amino acid having a changed structure) and may be described in terms of its attribute profile. As used herein, the term “attribute profile” refers to a listing of an antigen binding protein's attributes. In various instances, the attribute profile provides the chemical identity or attribute type, e.g., deamidation, optionally, relative to the native structure of the therapeutic protein. In various instances, the attribute profile provides the location of the attribute, e.g., the position of the amino acid on which the attribute is present. An attribute profile in some aspects, provides a description of all attributes present on the antigen binding protein. In other aspects, an attribute profile provides a description of a subset of attributes present on the protein. For example, an attribute profile may provide only those attributes that are present in a particular portion of the protein, e.g., the constant region, the variable region, the CDR. A species of a therapeutic protein such as an antibody or antigen binding protein is characterized by the attribute(s) present on the protein. A species of an antigen binding protein may differ from another species of the same protein by having a different attribute profile. When two therapeutic proteins have differing attribute profiles, the therapeutic proteins represent two different species of the therapeutic protein. When two therapeutic proteins have identical attribute profiles, the therapeutic proteins are considered as the same species of the therapeutic protein.

In various instances, the immunoglobulin, antibody or antigen binding protein is placed in a condition that leads to a change in its structure, e.g., formation of one or more attributes, and the change in structure alters the affinity of the therapeutic protein for its target. In various aspects, the immunoglobulin, antibody or antigen binding protein is placed in a condition that leads to a change in its structure, e.g., formation of one or more attributes, and the change in structure reduces the affinity of the antigen binding protein for its target. The reduced affinity in some aspects leads to a partial or total loss of the ability of the immunoglobulin, antibody or antigen binding protein to interact with (e.g., bind to) a target. In various instances, the partial or total loss of the ability of the immunoglobulin, antibody or antigen binding protein to interact with (e.g., bind to) a target ultimately reduces the antigen binding protein's efficacy. In alternative instances, the immunoglobulin, antibody or antigen binding protein is placed in a condition that leads to a change in its structure, e.g., formation of one or more attributes, and the change in structure does not alter the affinity of the immunoglobulin, antibody or antigen binding protein for its target. In various aspects, the change in structure does not reduce the affinity of the protein for its target. Without being bound to any particular theory, the methods of the present disclosure advantageously distinguish with precision and accuracy those attributes of an immunoglobulin, antibody or antigen binding protein that affect an interaction between the immunoglobulin, antibody or antigen binding protein and the target from attributes that do not affect the interaction.

In various aspects, a composition herein comprises a population of species of the immunoglobulin, antigen binding protein or fragment thereof, or antibody or fragment thereof. In various instances, the population is a homogenous population of the immunoglobulin, antigen binding protein or fragment thereof, or antibody or fragment thereof, optionally, each of the proteins present in the composition sample are the same species. In various instances, the population is a heterogeneous population comprising at least two different species of the immunoglobulin, antigen binding protein or fragment thereof, or antibody or fragment thereof having an attribute described herein. In various aspects, the heterogeneous population comprises at least 2, at least 3, at least 4, at least 5, at least 6 or more different species of the immunoglobulin, antigen binding protein or fragment thereof, or antibody or fragment thereof. Optionally, the heterogeneous population comprises more than 7, more than 8, more than 9, more than 10, more than 20, more than 30, more than 40, more than 50 different species of the protein. Each species of the population in some aspects has a unique attribute profile. In exemplary instances, the species of the immunoglobulin, antigen binding protein or fragment thereof, or antibody or fragment thereof are the only proteins present in the composition. In some aspects, the composition comprises (i) the population immunoglobulin, antigen binding protein or fragment thereof, or antibody or fragment thereof immunoglobulin, antigen binding protein or fragment thereof, or antibody or fragment thereof and (ii) a pharmaceutically-acceptable carrier, diluent, excipient, or a combination thereof. In some embodiments, at least 80%, 85%, 90%, 95%, or 99% of immunoglobulin, antigen binding protein or fragment thereof, or antibody or fragment thereof of the heterogeneous population comprises an attribute as described herein. In some embodiments, no more than 20%, 15%, 10%, 5%, or 1% of immunoglobulin, antigen binding protein or fragment thereof, or antibody or fragment thereof of the heterogeneous population comprises an attribute as described herein.

In exemplary embodiments, the method comprises applying a stress to an immunoglobulin, antigen binding protein or fragment thereof, or antibody or fragment thereof sample. In various instances, the stress may be any condition which leads to at least one change in structure of an amino acid of the immunoglobulin, antigen binding protein or fragment thereof, or antibody or fragment thereof or target, e.g., the stress may be any condition which leads to the formation of at least one attribute at an amino acid of the immunoglobulin, antigen binding protein or fragment thereof, or antibody or fragment thereof or target. Optionally, the stress leads to a change in structure in more than one amino acid of the immunoglobulin, antigen binding protein or fragment thereof, or antibody or fragment thereof or target, e.g., the stress leads to the formation or more than one attribute (e.g., at least or about 2, at least or about 3, at least or about 4, at least or about 5, at least or about 6, at least or about 7, at least or about 8, at least or about 9, at least or about 10, or more attributes). The stress in various instances leads to the formation of one or more attributes that are not present in the immunoglobulin, antigen binding protein or fragment thereof, or antibody or fragment thereof or target prior to the application of the stress. Accordingly, in some aspects, the application of stress leads to the formation of species of immunoglobulin, antigen binding protein or fragment thereof, or antibody or fragment thereof or target that were not present in the sample prior to the application of stress.

In exemplary aspects, the stress is an exposure to elevated temperatures to, e.g., 25 degrees C., 40 degrees C., 50 degrees C., optionally, in one or more buffers or formulations. In exemplary instances, such exposure to elevated temperatures mimics an accelerated stress program.

Optionally, the stress causes, about 5% to about 30%, about 10% to about 30%, about 15% to about 30%, about 20% to about 30%, about 25% to about 30%, about 5% to about 25%, about 5% to about 20%, about 5% to about 15%, or about 5% to about 10% of complexes formed between the immunoglobulin, antibody or antigen binding protein and the target to degrade or dissociate. In various aspects, the stress causes a reduced level of interactions between the immunoglobulin, antigen binding protein or fragment thereof, or antibody or fragment thereof and its target. In some aspects, the stress causes an about 10% to about 50% (e.g., about 10% to about 45%, about 10% to about 40%, about 10% to about 35%, about 10% to about 30%, about 10% to about 25%, about 10% to about 20%, about 10% to about 15%, about 10% to about 40%, about 10% to about 35%, about 10% to about 30%, about 10% to about 25%, about 10% to about 20%, or about 10% to about 15%) reduction in interactions, relative to interactions in corresponding conditions lacking the stress. In some aspects, the stress causes an increase in the K_{D} of the antibody or antigen binding protein for its target which K_{D} is associated with weaker binding. In some aspects, the stress causes a 10% to about 50% increase (e.g., about 10% to about 45%, about 10% to about 40%, about 10% to about 35%, about 10% to about 30%, about 10% to about 25%, about 10% to about 20%, about 10% to about 15%, about 10% to about 40%, about 10% to about 35%, about 10% to about 30%, about 10% to about 25%, about 10% to about 20%, or about 10% to about 15%) in the amount of unbound antibody or antigen binding protein. Without being bound to a particular theory, the stress applied in the presently disclosed methods leads to the generation of antibody or antigen binding protein species in a quicker and more robust, reproducible manner to obtain an abundance and variety of species for enhanced detection of species which might be created during manufacturing, storage and in human circulation (intravenous space or subcutaneous space in a human subject).

### Separation

In exemplary embodiments, the methods of the present disclosure comprise separating a mixture comprising different species of the antigen into at least two fractions. In some aspects, the mixture is separated into multiple (e.g., 2, 3, 4, 5, 6, 7, 8, 9, 10, or more) fractions. In some aspects, the separation step of the presently disclosed methods preserves native folding, high-order structure and binding ability of the antigen binding protein and its target. In various aspects, the mixture is separated into an unbound fraction comprises unbound antibody or antigen binding proteins or targets and a bound fraction comprises antibody/antigen binding protein-target complexes.

Suitable methods and techniques for separating mixtures into fractions are known in the art. See, e.g., Coskun, North Clin Istanb 3(2): 156-160 (2016); Snyder et al., Practical HPLC Method Development, 2nd ed., John Wiley & Sons, Inc. 1997; Snyder et al., Introduction to Modern Liquid Chromatography, John Wiley & Sons, Inc., Hoboken, NJ, 2010; Heftmann, Chromatography: Fundamentals and applications of chromatography and related differential migration methods, 6th ed., Volume 69A, Elsevier, Amsterdam, Netherlands, 2004; Mori and Barth, Size Exclusion Chromatography, Springer-Verlag, Berlin, 1999. In some aspects, the separation is based on charge, such as, e.g., ion exchange chromatography, capillary isoelectric focusing (cIEF) and/or capillary zone electrophoresis (CZE) or is based on hydrophobicity, such as, e.g., separation in reverse phase (RP; e.g., RP-HPLC) and hydrophobic interaction chromatography (HIC-HPLC). In various aspects, the separation is based on size such as, e.g., size exclusion chromatography (SEC; e.g., SE-HPLC), sodium dodecyl sulfate-polyacrylamide gel electrophoresis (SDS-PAGE), capillary electrophoresis with sodium dodecyl sulfate (CE-SDS). The methods described herein are used for detecting product oxidation of Met or Trp residues, fragmentation/clipping, isomerization of Asp, deamidation, formation of pyroglutamic acid at the N-terminus. In various embodiments, the mixture is separated into at least two fractions using a technique that separates components of a mixture based on size, charge, hydrophobicity, affinity for a capture molecule, or a combination thereof. In various instances, the technique is size exclusion chromatography (SEC), affinity chromatography, precipitation using beads or cells, free flow fractionation (FFF), ion exchange chromatography (IEX), cation exchange chromatography (CEX), hydrophobic interaction chromatography (HIC), or ultracentrifugation (UC). Optionally, the mixture is separated into at least two fractions using a technique that separates components of a mixture based on size, optionally, wherein the technique is size exclusion chromatography (SEC).

In various aspects, the mixture is separated into at least two fractions using a technique that separates components of a mixture based on affinity for a capture molecule bound to a solid support, optionally, a bead or a cell. In various instances, the mixture is separated by (i) adding the mixture to a container, e.g., a tube, comprising beads bound to the capture molecule or cells expressing at its surface the capture molecule, (ii) centrifuging the container (e.g., tube) to obtain a supernatant and a pellet, (iii) collecting the supernatant from the pellet to obtain the unbound fraction, (iv) releasing the bound fraction from the pellet with a solution, (v) centrifuging the container (e.g., tube) comprising the pellet and the solution to obtain a second supernatant comprising the bound fraction and a second pellet comprising the beads or cells, and (vi) collecting the second supernatant to obtain the bound fraction. The mixture in some aspects is separated by (i) adding the mixture to a column comprising beads bound to the capture molecule to obtain a flow-through and a bound fraction (ii) collecting the flow-through to obtain the unbound fraction, (iii) releasing the bound fraction from the beads with a solution and collecting the solution comprising the bound fraction. Suitable solid supports include, for example, beads, resin, paper, optionally, made of cellulose, silica, alumina, glass, plastic, or a combination thereof. In exemplary aspects the capture molecule bound to the solid support is a protein. The capture molecule may be identical to the target. Advantageously, the capture molecule is not limited to any particular molecule.

In various embodiments of the method of identifying attributes of an immunogolobuiln, antigen binding protein or target that affect an interaction between the antigen binding protein and the target, for each of the unbound fraction and bound fraction, the method comprises identifying and quantifying the abundance of each attribute present on a species of the antigen binding protein or target, wherein, when the abundance of an attribute in the unbound fraction is greater than the abundance of the attribute in the bound fraction, the attribute negatively affects the interaction between the antigen binding protein and the target. In various aspects, the method comprises using a mass spectrometer to identify and quantify the abundance of each attribute of the species of the antigen binding protein or target in each of the unbound fraction and bound fraction.

In various embodiments of the method of determining an effect of a known attribute present on a species of an antigen binding protein or target on an interaction between the antigen binding protein and the target, the method comprises for each of the unbound fraction and bound fraction, quantifying the abundance of the known attribute, wherein, when the abundance of the known attribute in the unbound fraction is greater than the abundance of the known attribute in the bound fraction, the known attribute has a negative effect on the interaction between the antigen binding protein and the target. In various aspects, the method comprises using a mass spectrometer to quantify the abundance of the known attribute in each of the unbound fraction and bound fraction.

Stability refers to resistance to chemical modifications of amino acid residues and biophysical protein modifications, such as formation of HMW species during stress conditions which may occur during manufacturing, storage and/or additional or alternative stress conditions. For methods and immunoglobulins, antigen binding proteins, and fragments thereof of embodiments described herein, “stability” and/or “HMW” species, may be determined using size exclusion chromatography (SEC). A composition comprising the immunoglobulin, antigen binding protein, or fragment may be separated by SEC, such as SEC-UV. The SEC may use a mobile phase comprising 100 mM sodium phosphate and 250 mM NaCl (pH 6.8), the flow rate may be set at 0.5 ml/min, the column temperature may be set at 37° C., the run time may be 35 minutes, and the auto sampler may be set at 4° C. An example of a suitable column for SEC includes a gel column comprising silica particles comprising a diol functional group and having a mean diameter of 5 μm and a mean pore size of about 25 nM (available commercially, for example, as a G3000SWxl column from TOSOH Bioscience). For SEC-UV, ultraviolet/Visible spectrometry (UV/VIS) detection may be performed at 214 nm and 280 nm. It will be appreciated that following separation, peaks representing the monomer and HMW species can elute at different times in the SEC elution profile.

In the case of determinations of stability, the composition for the SEC analysis may comprise stressed immunoglobulin, antigen binding protein, or fragment, which may be stressed at an elevated temperature for a period of time, such as 40° C. for four weeks. It is noted that 40° C. for four weeks generally extrapolates well to shelf life stability for immunoglobulins, antigen binding proteins, and fragments thereof (shelf life stability is typically 2 years at 2-8° C. (2Y4C) followed by 1 month at room temperature, which is 25° C. or 30° C. depending on geographic location). Additionally or alternatively, ultraviolet light (klux/hr cool white light and 10 W/m² UVA light at 25° C. for 7 days), extreme pH (pH≥8 or ≤3.6), or oxidizing reagents (e.g., 0.1% H₂O₂ at 25° C. for 5 hours) may be used as stressors. Unless stated otherwise herein or necessitated otherwise by scientific context, stress for the purposes of “stability” will be understood to refer to 40° C. for four weeks. Additional information on stressors and SEC analysis may be found, for example in International Pub. No. WO 2020/247790, which is hereby incorporated by reference in its entirety.

Following SEC analysis, peptide mapping may optionally be performed, and peptide modifications associated with bound and unbound species may be identified, for example as described herein and/or in International Pub. No. WO 2020/247790. For peptide mapping, the eluting fractions may be collected using a filter with a molecular weight cut-off (for example, greater than 10 kDa) and eluted with a 7.5 M guanidine elution buffer. To determine chemical modifications affecting binding to antigen, stressed immunoglobulin (or antigen binding protein or fragment thereof) and antigen may be mixed together and separated on earlier eluting antigen-bound complex and later eluting unbound immunoglobulin (or antigen binding protein or fragment thereof). To determine chemical modifications impacting or correlating with HMW, monomeric and HMW species may be collected. In the described study, thermal forced stress and related degradations of the antibody at 40 C for 1 month (40C1M) were used. The stressed antibody was mixed with its target (TSLP), and the mixture was separated by SEC on antibody-target complex and unbound antibody. Two limitations of this applied SEC antibody-antigen method should be noted. The 40C1M stress may lead to greater degradations as compared to the room temperature degradations at 25 C or 30 C. Also, a degradation/modification on one residue may cause modification on another residue by long range, allosteric interactions. This effect can increase at higher temperature, since the structure will be more amenable to dynamic motion. Also, the described SEC antigen-antibody method analyzes all species in the samples. This is different from the conventional method including CEX separation on individual peaks followed by characterization, where the collected species are better defined and species “between the main peaks” with two and more modifications per molecule are avoided.

It will be appreciated that “affinity” or “binding” may be determined by surface plasmon resonance (SPR), bio-layer interferometry, or also by SEC binding affinity experiments as described herein. Unless stated otherwise herein or necessitated otherwise by scientific context, “affinity” will be understood to refer to affinity as measured by SPR. Kd value may be measured by SPR using a biosensor system such as a BIAcore® system. The analysis with the BIAcore® system may comprise analyzing the binding and dissociation of an antigen (e.g., TSLP) from chips with immobilized molecules (e.g., anti-TSLP immunoglobulin, antigen binding protein, or fragment thereof as described herein) on their surface. Binding complexes with Kd<10⁻⁶M can be detected using SPR. In various embodiments, the SPR may be carried out at 20°, 25°, 30° or 37° C.

### Compositions

In various embodiments, provided is a composition comprising a plurality of anti-TSLP immunoglobulins, antigen binding proteins or fragments thereof, or antibodies or fragments thereof each comprising: a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ IDNO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO: 8, comprising at least one of: L-aspartate at HC position 54 of SEQ ID NO: 7, which comprises neither isoAspartate (isoAsp) nor cyclic aspartate (cAsp); non-oxidized HC W102 of SEQ ID NO: 8; L-aspartate at LC position 49 or position 50 of SEQ ID NO: 7, which comprises neither isoAsp nor cAsp; LC N65 set out in SEQ ID NO: 12 which does not comprise deamidated N65; or L-aspartate at LC position 91 of SEQ ID NO: 5, which comprises neither isoAsp nor cAsp

In various embodiments, provided is a composition comprising a plurality of anti-TSLP monoclonal antibodies or antigen binding fragments thereof each comprising: a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ IDNO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO: 8, comprising at least one of: L-aspartate at HC position 54 of SEQ ID NO: 7, which comprises neither isoAspartate (isoAsp) nor cyclic aspartate (cAsp); non-oxidized HC W102 of SEQ ID NO: 8; L-aspartate at LC position 49 or position 50 of SEQ ID NO: 4, which comprises neither isoAsp nor cAsp; deamidated LC N65 of SEQ ID NO: 12; or L-aspartate at LC position 91 of SEQ ID NO: 5, which comprises neither isoAsp nor cAsp.

In various embodiments, no more than 0.9% of the anti-TSLP monoclonal antibodies comprise isomerized HC D54. In various embodiments, no more than 2% of the anti-TSLP monoclonal antibodies comprise oxidized HC W102. In various embodiments, no more than 0.9% of the anti-TSLP monoclonal antibodies comprise isomerized LC D50. In various embodiments, no more than 0.5% of the anti-TSLP monoclonal antibodies comprise deamidated LC N65. In various embodiments, no more than 0.9% of the anti-TSLP monoclonal antibodies comprise isomerized HC D91. In various embodiments, the anti-TSLP antibody comprises a combination of L-aspartate at HC 54 and L-aspartate at LC 49 or 50. In various embodiments, the anti-TSLP antibody is enriched in L-aspartate at HC54 to at least 6-fold over the levels of isoAsp. In various embodiments, the anti-TSLP antibody is an IgG2 antibody.

In one aspect, the composition comprises an anti-TSLP immunoglobulin, antigen binding protein or fragment thereof, or antibody or fragment thereof comprising (A) a light chain variable domain comprising: (i) a light chain CDR1 sequence comprising the amino acid sequence set forth in SEQ ID NO: 3; (ii) a light chain CDR2 sequence comprising the amino acid sequence set forth in SEQ ID NO: 4; and (iii) a light chain CDR3 sequence comprising the amino acid sequence set forth in SEQ ID NO: 5; and (B) a heavy chain variable domain comprising: (i) a heavy chain CDR1 sequence comprising the amino acid sequence set forth in SEQ ID NO: 6; (ii) a heavy chain CDR2 sequence comprising an amino acid sequence with a mutation at at least one of the following residues, D54 or G55 set forth in SEQ ID NO: 7, and (iii) a heavy chain CDR3 sequence comprising the amino acid sequence set forth in SEQ ID NO:8. In various embodiments, the HCDR2 has the sequence VIWYX₁X₂SNKHYADSVKG, wherein X₁ is D or E and X₂ is G or A (SEQ ID NO: 13). Optionally the HCDR2 has the following sequence: VIWYEGSNKHYADSVKG (SEQ ID NO: 14), VIWYDASNKHYADSVKG (SEQ ID NO: 15) or VIWYEASNKHYADSVKG (SEQ ID NO: 16).

In various embodiments, the mutation in HCDR2 is D54E. In various embodiments, the mutation in HCDR2 is G55A. In various embodiments, the anti-TSLP antigen binding protein or fragment thereof optionally comprises a mutation in at least one of the following residues of LCDR2 D49, D50, or S51 of SEQ ID NO: 4. In various embodiments, the mutation of LCDR2 is one or more of D49E, D50E, or S51A. In various embodiments, the LCDR2 has the sequence X₁X₂X₃DRPS, wherein X₁ is D or E, X₂ is D or E, and X₃ is S or A (SEQ ID NO: 17). Optionally the LCDR2 has the following sequence: EDSDRPS (SEQ ID NO: 18), DESDRPS (SEQ ID NO: 19), EESDRPS (SEQ ID NO: 20), DDADRPS (SEQ ID NO: 21), DEADRPS (SEQ ID NO: 22), EDADRPS (SEQ ID NO: 23) or EEADRPS (SEQ ID NO: 24).

In various embodiments, the composition comprises an anti-TSLP immunoglobulin, antigen binding protein or fragment thereof, or antibody or fragment thereof comprising (A) a light chain variable domain comprising: (i) a light chain CDR1 sequence comprising the amino acid sequence set forth in SEQ ID NO: 3; (ii) a light chain CDR2 sequence comprising an amino acid sequence with a mutation in at least one of the following residues D49, D50, or S51 of SEQ ID NO: 4; and (iii) a light chain CDR3 sequence comprising the amino acid sequence set forth in SEQ ID NO: 5; and (B) a heavy chain variable domain comprising: (i) a heavy chain CDR1 sequence comprising the amino acid sequence set forth in SEQ ID NO: 6; (ii) a heavy chain CDR2 sequence comprising the amino acid sequence set forth in SEQ ID NO: 7 and (iii) a heavy chain CDR3 sequence comprising the amino acid sequence set forth in SEQ ID NO: 8.

In various embodiments, the LCDR2 has the sequence X₁X₂X₃DRPS (SEQ ID NO: 17), wherein X₁ is D or E, X₂ is D or E, and X₃ is S or A. Optionally the LCDR2 has the following sequence: EDSDRPS (SEQ ID NO: 18), DESDRPS (SEQ ID NO: 19), EESDRPS (SEQ ID NO: 20), DDADRPS (SEQ ID NO: 21), DEADRPS (SEQ ID NO: 22), EDADRPS (SEQ ID NO: 23) or EEADRPS (SEQ ID NO: 24). In various embodiments, the mutation in LCDR2 is D49E. In various embodiments, the mutation in LCDR2 is D50E. In various embodiments, the mutation in LCDR2 is S51A. In various embodiments, the anti-TSLP immunoglobulin, antigen binding protein or fragment thereof, or antibody or fragment thereof optionally comprises a mutation at one of the following residues D54 or G55 in HCDR2 set out in SEQ ID NO: 7. In various embodiments, the mutation in HCDR2 is one or more of D54E or G55A in SEQ ID NO: 7. In various embodiments, the HCDR2 has the sequence VIWYX₁X₂SNKHYADSVKG, wherein X₁ is D or E and X₂ is G or A (SEQ ID NO: 13). Optionally the HCDR2 has the following sequence: VIWYEGSNKHYADSVKG (SEQ ID NO: 14), VIWYDASNKHYADSVKG (SEQ ID NO: 15) or VIWYEASNKHYADSVKG (SEQ ID NO: 16).

In various embodiments, the composition comprises an anti-TSLP immunoglobulin, antigen binding protein or fragment thereof, or antibody or fragment thereof comprising (A) a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; or iii. a sequence of amino acids encoded by a polynucleotide that hybridizes under moderately stringent conditions to the complement of a polynucleotide consisting of SEQ ID NO:11; or (B) a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; or iii. a sequence of amino acids encoded by a polynucleotide that hybridizes under moderately stringent conditions to the complement of a polynucleotide consisting of SEQ ID NO:9; or (C) a light chain variable domain of (A) and a heavy chain variable domain of (B), wherein the anti-TSLP immunoglobulin, antigen binding protein or fragment thereof, or antibody or fragment thereof retains one or more of CDRs of the anti-TSLP antigen binding proteins or fragment thereof and comprises a mutation at one or more of HCDR2 D54 or G55 of SEQ ID NO: 7, or LCDR2 D49, D50, or S51 of SEQ ID NO: 4.

In various embodiments, the anti-TSLP immunoglobulin, antigen binding protein or fragment thereof, or antibody or fragment thereof comprises a heavy chain comprising the amino acid sequence of

(HCDR2 is underlined)
(SEQ ID NO: 25)
QMQLVESGGGVVQPGRSLRLSCAASGFTFRTYGMHWVRQAPGKGLEWVAV
IWYX₁X₂SNKHYADSVKGRFTITRDNSKNTLNLQMNSLRAEDTAVYYCAR
APQWELVHEAFDIWGQGTMVTVSS

wherein X₁ is D or E and X₂ is G or A, optionally

(SEQ ID NO: 26)
QMQLVESGGGVVQPGRSLRLSCAASGFTFRTYGMHWVRQAPGKGLEWVAV
IWYEGSNKHYADSVKGRFTITRDNSKNTLNLQMNSLRAEDTAVYYCARAP
QWELVHEAFDIWGQGTMVTVSS;
or,
(SEQ ID NO: 27)
QMQLVESGGGVVQPGRSLRLSCAASGFTFRTYGMHWVRQAPGKGLEWVAV
IWYDASNKHYADSVKGRFTITRDNSKNTLNLQMNSLRAEDTAVYYCARAP
QWELVHEAFDIWGQGTMVTVSS;
or,
(SEQ ID NO: 28)
QMQLVESGGGVVQPGRSLRLSCAASGFTFRTYGMHWVRQAPGKGLEWVAV
IWYEASNKHYADSVKGRFTITRDNSKNTLNLQMNSLRAEDTAVYYCARAP
QWELVHEAFDIWGQGTMVTVSS,
or
mixtures thereof.

In various embodiments, the anti-TSLP immunoglobulin, antigen binding protein or fragment thereof, or antibody or fragment thereof comprises a light chain comprising the amino acid sequence of

(LCDR1-3 are underlined)
(SEQ ID NO: 29)
SYVLTQPPSVSVAPGQTARITCGGNNLGSKSVHWYQQKPGQAPVLVVYX₁
X₂X₃DRPSWIPERFSGSNSGNTATLTISRGEAGDEADYYCQVWDSSSDHV
VFGGGTKLTVL,

wherein X₁ is D or E, X₂ is D or E, and X₃ is S or A, optionally

(SEQ ID NO: 30)
SYVLTQPPSVSVAPGQTARITCGGNNLGSKSVHWYQQKPGQAPVLVVYED
SDRPSWIPERFSGSNSGNTATLTISRGEAGDEADYYCQVWDSSSDHVVFG
GGTKLTVL;
or,
(SEQ ID NO: 31)
SYVLTQPPSVSVAPGQTARITCGGNNLGSKSVHWYQQKPGQAPVLVVYDE
SDRPSWIPERFSGSNSGNTATLTISRGEAGDEADYYCQVWDSSSDHVVFG
GGTKLTVL;
or,
(SEQ ID NO: 32)
SYVLTQPPSVSVAPGQTARITCGGNNLGSKSVHWYQQKPGQAPVLVVYEE
SDRPSWIPERFSGSNSGNTATLTISRGEAGDEADYYCQVWDSSSDHVVFG
GGTKLTVL;
or,
(SEQ ID NO: 33)
SYVLTQPPSVSVAPGQTARITCGGNNLGSKSVHWYQQKPGQAPVLVVYDD
ADRPSWIPERFSGSNSGNTATLTISRGEAGDEADYYCQVWDSSSDHVVFG
GGTKLTVL;
or,
(SEQ ID NO: 34)
SYVLTQPPSVSVAPGQTARITCGGNNLGSKSVHWYQQKPGQAPVLVVYDE
ADRPSWIPERFSGSNSGNTATLTISRGEAGDEADYYCQVWDSSSDHVVFG
GGTKLTVL;
or,
(SEQ ID NO: 35)
SYVLTQPPSVSVAPGQTARITCGGNNLGSKSVHWYQQKPGQAPVLVVYED
ADRPSWIPERFSGSNSGNTATLTISRGEAGDEADYYCQVWDSSSDHVVFG
GGTKLTVL;
or,
(SEQ ID NO: 36)
SYVLTQPPSVSVAPGQTARITCGGNNLGSKSVHWYQQKPGQAPVLVVYEE
ADRPSWIPERFSGSNSGNTATLTISRGEAGDEADYYCQVWDSSSDHVVFG
GGTKLTVL;
or
mixtures thereof.

Also provided is a composition comprising anti-TSLP monoclonal antibodies each comprising a TSLP antibody having the sequences described herein, e.g., one or more CDRs set out in SEQ ID NO: 3-8 and SEQ ID NOs: 13-24 and one or more variable regions set out in SEQ ID NO: 10 and 12 and SEQ ID NOs: 25-36, the composition comprising a limited content of isomerized HC D54 and/or a limited content of isomerized LC D49 or D50, effective for the anti-TSLP monoclonal antibodies of the composition to bind to TLSP with a Kd that is numerically less than or equal to 10⁻⁸ M. In various embodiments, an anti-TSLP antibody described herein binds at least with an affinity (Kd) of 10⁻⁸ M, 10⁻⁹ M, 10⁻¹⁰ M, 10⁻¹¹ M, 10⁻¹² M, 10⁻¹³ M or less.

Also provided is a composition comprising anti-TSLP monoclonal antibodies each comprising a TSLP antibody having the sequences described herein, e.g., CDRs set out in SEQ ID NOs: 3-8, or SEQ ID NOs: 13-24 and/or variable regions set out in SEQ ID NO: 10 and 12 or SEQ ID NOs: 25-36, the composition comprising IgG2 anti-TSLP monoclonal antibodies, wherein at least one of: no more than 0.9% of the anti-TSLP monoclonal antibodies comprise isomerized HC D54; no more than 2% of the anti-TSLP monoclonal antibodies comprise oxidized HC W102; no more than 0.9% of the anti-TSLP monoclonal antibodies comprise isomerized LC D50; no more than 0.5% of the anti-TSLP monoclonal antibodies comprise deamidated LC N65; or no more than 0.9% of the anti-TSLP monoclonal antibodies comprise isomerized LC D91.

In some embodiments, the composition is part of a formulation described herein. In some embodiments, the composition is a drug substance used to produce a formulation as described herein.

### Methods of Administration

In one aspect, methods of the present disclosure include a step of administering a therapeutic anti-TSLP antibody or antibody variant described herein, optionally in a pharmaceutically acceptable carrier or excipient. In certain embodiments, the pharmaceutical composition is a sterile composition.

Contemplated herein are methods for treating an inflammatory disease, condition or disorder, such as asthma, chronic obstructive pulmonary disease (COPD), atopic dermatitis, eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF) with an anti-TSLP antibody or antigen binding protein or fragments thereof as described herein. In various embodiments, the disease, condition or disorder is asthma, including severe asthma, eosinophilic or non-eosinophilic asthma and low eosinophil asthma.

Asthma is a chronic inflammatory disorder of the airways. Each year, asthma accounts for an estimated 1.1 million outpatient visits, 1.6 million emergency room visits, 444,000 hospitalizations (Defrances et al, 2008) Available at: the Centers for Disease Control website, www.cdc.gov/nchs/data/nhsr/nhsr005.pdf, and 3,500 deaths in the U.S. In susceptible individuals, asthmatic inflammation causes recurrent episodes of wheezing, breathlessness, chest tightness, and cough. The etiology of asthma is thought to be multi-factorial, influenced by both genetic environmental mechanisms (To et al., BMC Public Health 2012; 12:204; Chung et al. Eur Respir J 2014; 43:343-73), with environmental allergens an important cause (Chung et al., supra; Pavord I D, et al., NPJ Prim Care Respir Med 2017; 27:17). The majority of cases arise when a person becomes hypersensitive to allergens (atopy). Atopy is characterized by an increase in Th2 cells and Th2 cytokine expression and IgE production. Approximately 10 million patients in the United States are thought to have allergy-induced asthma. Despite the available therapeutic options, asthma continues to be a major health problem. Worldwide, asthma currently affects approximately 300 million people; by 2020, asthma is expected to affect 400 million people (Partridge, Eur Resp Rev. 16:67-72, 2007).

Allergen inhalation by atopic asthmatics induces some of the manifestations of asthma, including reversible airflow obstruction, airway hyperresponsiveness, and eosinophilic and basophilic airway inflammation. Allergen inhalation challenge has become the predominant model of asthma in many species (Bates et al., Am J Physiol Lung Cell Mol Physiol. 297(3): L401-10, 2009; Diamant et al., J Allergy Clin Immunol. 132(5):1045-1055, 2013.)

Different asthma subtypes that are refractory to steroid treatment have been identified. Eosinophils are important inflammatory cells in allergic asthma that is characteristically mediated by Th2-type CD4+ T cells. Neutrophilic airway inflammation is associated with corticosteroid treatment in severe asthma and can be mediated by Th1- or Th17-type T cells (Mishra et al., Dis. Model. Mech. 6:877-888, 2013).

Measures of diagnosis and assessment of asthma include the following: Airway inflammation evaluated using a standardized single-breath Fraction of Exhaled Nitric Oxide (FeNO)(American Thoracic Society; ATS, Am J Respir Crit Care Med. 171(8):912-30, 2005) test. Spirometry is performed according to ATS/European Respiratory Society (ERS) guidelines (Miller et al, Eur Respir J. 26(1):153-61, 2005). Post-bronchodilator (Post-BD) spirometry testing is assessed after the subject has performed pre-BD spirometry. Maximal bronchodilation is induced using a SABA such as albuterol (90 μg metered dose) or salbutamol (100 μg metered dose) or equivalent with a spacer device for a maximum of 8 total puffs (Sorkness et al, J Appl Physiol. 104(2):394-403, 2008). The highest pre- and post-BD FEV₁ obtained after 4, 6, or 8 puffs is used to determine reversibility and for analysis. Asthma Control Questionnaire (ACQ) 6 is a patient-reported questionnaire assessing asthma symptoms (i.e., night-time waking, symptoms on waking, activity limitation, shortness of breath, wheezing) and daily rescue bronchodilator use and FEV₁ (Juniper et al, October 1999). The ACQ-6 is a shortened version of the ACQ that omits the FEV₁ measurement from the original ACQ score. The mean ACQ score is the mean of the responses. Mean scores of ≤0.75 indicate well-controlled asthma, scores between 0.75 and ≤1.5 indicate partly-controlled asthma, and a score >1.5 indicates uncontrolled asthma (Juniper et al, Respir Med. 100(4):616-21, 2006). Individual changes of at least 0.5 are considered to be clinically meaningful (Juniper et al, Respir Med. 99(5):553-8, 2005). The Asthma Quality of Life Questionnaire, Standardized (AQLQ[S])+12 (AQLQ(S)+12) is a 32-item questionnaire that measures the HRQoL experienced by asthma patients (Juniper et al, Chest. 115(5): 1265-70, May 1999). The Asthma Daily Diary is also used for assessment.

Related US Patent Publication US-2018-0296669 (incorporated herein by reference) discloses that treatment with an anti-TSLP antibody is effective at reducing asthma symptoms in a no eosinophil/low eosinophil population as it is in a high eosinophil population. Also contemplated is a method of reducing the frequency of asthma exacerbation in a subject.

Also contemplated herein are methods of treating asthma in a subject having a Th2 high asthma profile or a Th2 low asthma profile. It is contemplated that a TSLP antagonist that inhibits binding of the TSLP protein to its receptor complex will effectively treat a low eosinophil asthma population as the antibody described herein. Similarly, it is contemplated that a TSLP antagonist that inhibits binding of TSLP to its receptor complex will be effective in treating Th2 low asthma populations. Also contemplated are methods for treating chronic obstructive pulmonary disease (COPD) in a subject comprising administering an anti-TSLP antibody or antibody variant or antigen binding protein described herein. It is contemplated that the subject to be treated is human. The subject may be an adult, an adolescent or a child.

Therapeutic antibody (or antibody variant) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, or at a longer interval.

In various embodiments, the amounts of therapeutic agent, such as a bivalent antibody having two TSLP binding sites, in a given dosage may vary according to the size of the individual to whom the therapy is being administered as well as the characteristics of the disorder being treated.

In exemplary treatments, the anti-TSLP antibody or antibody variant is administered in a dose range of about 70 mg to about 280 mg per daily dose. For example, the dose may be given in about 70 mg, 210 mg or 280 mg. In various embodiments, the anti-TSLP antibody or antibody variant may be administered at a dose of 70, 80, 90, 100, 110, 120, 130, 140, 150, 160, 10, 180, 190, 200, 210, 220, 230, 240, 250, 260, 270 or 280 mg per dose. These concentrations may be administered as a single dosage form or as multiple doses. The above doses are given every two weeks or every four weeks. In various embodiments, the anti-TSLP antibody or antibody variant is administered at a single dose of 70 mg every two weeks or every four weeks. In various embodiments, the anti-TSLP antibody or antibody variant is administered at a single dose of 210 mg every two weeks or every four weeks. In various embodiments, the anti-TSLP antibody or antibody variant is administered at a single dose of 280 mg every two weeks or every four weeks.

For antibody variants, the amount of antibody variant should be such that the number of TSLP binding sites that are in the dose have an equimolar number of TSLP binding sites to canonical bivalent antibody described above.

It is contemplated that the anti-TSLP antibody or antibody variant is administered every 2 weeks or every 4 weeks for a period of at least 4 months, 6 months, 9 months, 1 year or more. In various embodiments, the administration is subcutaneous or intravenous.

Treatment with the anti-TSLP antibody or antibody variant is contemplated to decrease eosinophils in blood, sputum, broncheoalveolar fluid, or lungs of the subject. It is also contemplated that the administration shifts cell counts in the subject from a Th2 high population to a Th2 low population. It is further contemplated that administration of the anti-TSLP antibody improves one or more measures of asthma in a subject selected from the group consisting of forced expiratory volume (FEV), FEV1 reversibility, forced vital capacity (FVC), FeNO, Asthma Control Questionnaire-6 score and AQLQ(S)+12 score.

Improvement in asthma may be measured as one or more of the following: reduction in AER (annualized exacerbation rate), reduction in hospitalizations/severe exacerbations for asthma, change from baseline (increase) in time to first asthma exacerbation (following onset of treatment with anti-TSLP antibody), decrease relative to placebo in proportion of subjects with one or more asthma exacerbations or severe exacerbations over the course of treatment, e.g., 52 weeks, change from baseline (increase) in FEV1 and FVC (pre-broncholdilator and post-bronchodilator), change from baseline (decrease) in blood or sputum eosinophils (or lung eosinophils if biopsy or BAL fluid obtained), change from baseline (decrease) in FeNO, change from baseline (decrease) in IgE, improvement in asthma symptoms and control as measured by PROs including ACQ and variants, AQLQ and variants, SGRQ, and asthma symptom diaries, change (decrease) in use of rescue medications, decrease in use of systemic corticosteroids, decrease in Th2/Th1 cell ratio in blood. Most/all these measures should be in total population and subpopulations including hi and low eosinophils (Greater than or equal to 250 is high; less than 250 is low), allergic and non-allergic, Th2 hi and low, Periostin hi and low (compared to median value), and FeNO hi and low (greater than or equal to 24 or less than 24).

Also contemplated in the present disclosure is the administration of multiple agents, such as an antibody composition in conjunction with a second agent as described herein, including but not limited to an anti-inflammatory agent or asthma therapy.

However, it is contemplated that, in various embodiments, the administration reduces frequency of or levels of co-administered therapy in the subject. Exemplary co-administered therapies include, but are not limited to, inhaled corticosteroids (ICS), long-acting β2 agonist (LABA), leukotriene receptor antagonists [LTRA], long-acting anti-muscarinics [LAMA], cromones, short-acting β2 agonist (SABA), and theophylline or oral corticosteroids. In various embodiments, the administration eliminates the need for corticosteroid therapy.

### Formulations

In some embodiments, the disclosure contemplates use of pharmaceutical compositions comprising a therapeutically effective amount of an anti-TSLP antibody or antibody variant together with a pharmaceutically acceptable diluent, carrier, solubilizer, emulsifier, preservative, and/or adjuvant. In addition, the disclosure provides methods of treating a subject by administering such pharmaceutical composition.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

A suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor.

The formulation components are present preferably in concentrations that are acceptable to the site of administration. In certain embodiments, buffers are used to maintain the composition at physiological pH or at a slightly lower pH, typically within a pH range of from about 4.5 to about 8. Including about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0.

In various embodiments, the anti-TSLP antibody or antibody variant is in a formulation containing acetate, and one or more of proline, sucrose, polysorbate 20 or polysorbate 80. In various embodiments, the formulation comprises 5-50 mM acetate, less than or equal to 3% (w/v) proline, 0.015% (w/v)±0.005% (w/v) polysorbate 20 or polysorbate 80, at pH between 4.9 and 6.0. Optionally, the antibody or antibody fragment is at a concentration of between about 100 and about 150 mg/ml. The formulation may be stored at −20° to −70° C. Exemplary anti-TSLP formulations comprising these excipients are described in International Application No. PCT/US2021/018561, herein incorporated by reference.

In alternative embodiments, the anti-TSLP antibody or antibody variant is in a formulation containing a surfactant, and at least one basic amino acid or a salt thereof. In exemplary instances, the basic amino acid is arginine or histidine. In various embodiments, the salt is arginine glutamate or histidine glutamate, optionally in a concentration of from 10 to 200 mM. Optionally, the formulation further comprises proline. In alternative embodiments, the anti-TSLP antibody or antibody variant is in a formulation containing a surfactant, and calcium or a salt thereof. In various embodiments, the salt is calcium glutamate, optionally in a concentration from 15 mM to about 150 mM. Optionally, the formulation further comprises proline. In various embodiments, the surfactant is polysorbate 20 or polysorbate 80 or a mixture thereof. Optionally, the antibody or antibody fragment is at a concentration of greater than about 110 mg/ml, or greater than about 140 mg/ml. Exemplary anti-TSLP formulations comprising these excipients are described in International Patent Application No. PCT/US2021/017880, herein incorporated by reference.

When parenteral administration is contemplated, the therapeutic compositions for use may be provided in the form of a pyrogen-free, parenterally acceptable aqueous solution comprising the desired anti-TSLP antibody in a pharmaceutically acceptable vehicle. A particularly suitable vehicle for parenteral injection is sterile distilled water in which the antibody is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the antibody. In various embodiments, the administration may be via pre-filled syringe or autoinjector. In various embodiments, the auto-injector is an Ypsomed YpsoMate® device. In various embodiments, the auto-injector is disclosed in WO 2018/226565, WO 2019/094138, WO 2019/178151, WO 20120/072577, WO2020/081479, WO 2020/081480, PCT/US20/70590, PCT/US20/70591, PCT/US20/53180, PCT/US20/53179, PCT/US20/53178, or PCT/US20/53176.

### Kits

As an additional aspect, the disclosure includes kits which comprise one or more compounds or compositions packaged in a manner which facilitates their use to practice methods of the disclosure. In one embodiment, such a kit includes a compound or composition described herein, packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the antibody composition.

Additional aspects and details of the disclosure will be apparent from the following examples, which are intended to be illustrative rather than limiting.

### EXAMPLES

### Example 1

Tezepelumab (AMG157) was tested for its stability and ability to form HMW species at high stress temperatures. Tezepelumab was subjected to temperature stress conditions, taking antibody in formulation at 37° C. and increasing the temperature to the conditions as described below. Attributes impacting binding and stability were determined using size exclusion chromatography and peptide mapping.

### Materials and Methods

AMG 157 and labile residues potentially impacting binding: Amino acid sequence of AMG157 as sequence A5 (and as chains H5, L5) and also several other TSLP-binding antibodies were previously described in patent U.S. Pat. No. 7,982,016 B2.

Molecular mass of the antibody with A2G0F/A2G0F glycosylation (C6500 H9998 O2068 N1734 S52) is 147189.4 Da, including heavy chain N-terminal pyroglutamate and C-terminal K removed. TSLP contained 74% monomeric, 23% dimeric and 3% tetrameric species.

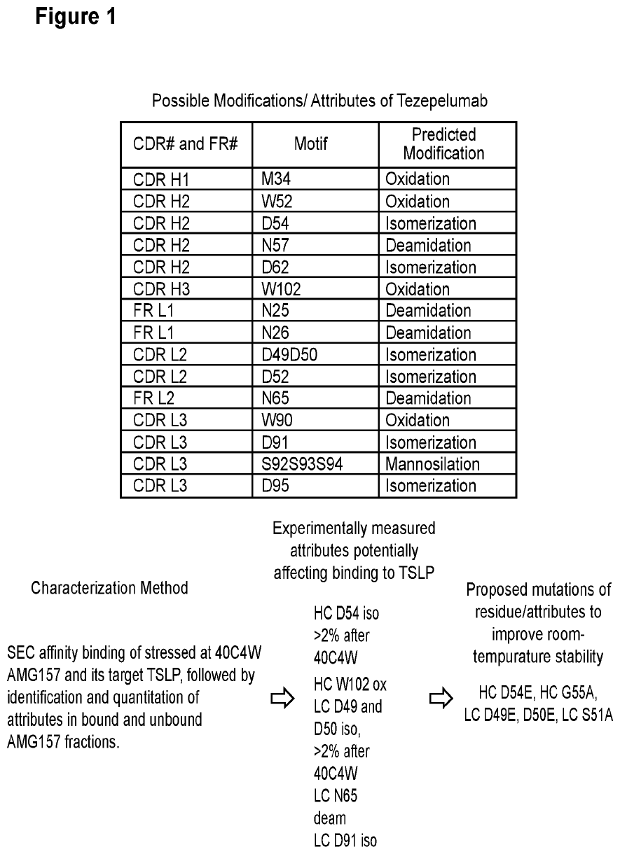

In silico assessment of the sequence following the Molecule Assessment identified several residues in CDRs, which are potentially susceptible to chemical modifications that may impact binding and potency. Those CDR residues as well as several other residues from frameworks were considered, and they include aspirational target ranges based on current understanding (FIG. 1, top). The residues in CDRs and their common modifications are selected as possible attributes, because they can potentially affect the binding to target and potency.

Method for identification of chemical modifications impacting binding; Size Exclusion Chromatography (SEC) and fraction Collection: After incubation, the AMG157 mixture was separated by SEC using a G3000SWxl TOSOH Bioscience, 7.8 mm ID×30 cm column (Catalog #08541, TOSOH Bioscience, San Francisco, CA) and the mobile-phase included 100 mM sodium phosphate and 250 mM NaCl (pH 6.8). The flow rate was set at 0.5 ml/min, the column temperature was set at 37° C., the run time was 35 minutes, and the auto sampler was set at 4° C. Ultraviolet/Visible spectrometry (UV/VIS) detection was performed at 214 nm and 280 nm. The eluting fractions were collected using a filter with a molecular weight cut-off of above 10 kDa and eluted with a 7.5 M guanidine elution buffer. The eluted fractions were subjected to sample preparation for peptide mapping described below.

SEC of antibody with ligand complex followed by LC-MS/MS characterization determines the ratio of modifications in unbound and bound fractions of antibody. This method is different than an SEC method that typically detects aggregation of proteins, e.g., differentiating between monomers and dimers, etc., since it detects binding between antibody and ligand not just aggregation of the antibody itself. The SEC binding affinity experiment was initiated by mixing AMG157 protein with its target. Upon the separation of the antibody-antigen mixtures by SEC-UV, peaks representing the bound complex of therapeutic protein, the ligand, and the unbound therapeutic protein containing attributes eluted at different time in the SEC elution profile. This allowed collection of the fractions of the bound antibody-antigen complex and the unbound antibody. Once the collected fractions are digested by trypsin and analyzed using LC-MS/MS method, abundance plots of the attributes of therapeutic proteins in the bound and unbound fractions were generated. A volcano plot was also generated with log 2 fold change as x-axis and −log 10 p-value as the y-axis. Log 2 fold change represents the ratio of the attributes in unbound/bound fractions, which indicates how much the attribute impacts the binding of therapeutic protein to ligand. Minus log 10 of p-value represents how confident the fold of change is represented.

Method for identification of chemical modifications impacting aggregation at 50° C.: A similar approach was used to study the attributes in the high molecular weight (HMW) species and monomer species in the 50° C.1W sample of AMG 157. SEC-UV of AMG 157 50° C.1W followed by LC-MS/MS characterization determines the ratio of modifications in HMW and monomer fractions of antibody. Upon the SEC separation, peaks representing the monomer and HMW species with identified attributes can elute at different time in the SEC elution profile. The HWW and monomer fractions of AMG 157 50° C.1W were collected, digested, and analyzed using LC-MS/MS methods. Abundance plots of the attributes of therapeutic proteins in HMW and monomer antibody were generated. A volcano plot was also generated with log 2 fold change as x-axis and −log 10 p-value as the y-axis. log 2 fold change represents the ratio of the attributes in HMW/monomer fractions, which indicates how much the attribute causes the formation of HMW antibody. Minus log 10 of p-value represents how confident the fold of change is.

Peptide Mapping: Peptide mapping of the collected fractions was performed using the sample preparation procedure including refolding with guanidine, reduction and alkylation of disulfide bonds, buffer exchange and digestion with trypsin on peptides suitable for LC-MS analysis as described in (Ren et al., Anal. Biochem. 392: 12-21 (2009)). Briefly, a sample comprising AMG157 was diluted to about 1 mg/ml in 0.5 ml of pH 7.5 denaturation buffer (7.5 M guanidine hydrochloride (GdnHCl) and 0.25 M Tris). Reduction was accomplished with the addition of 3 μl of 0.5 M dithiothreitol (DTT) followed by 30 min of incubation at room temperature. Carboxy-methylation was achieved with the addition of 7 μl of 0.5 M iodoacetic acid (IAA). The reaction was carried out in the dark for 15 min at room temperature. Excess IAA was quenched with the addition of 4 μl of 0.5 M DTT. Reduced and alkylated AMG157 samples were buffer-exchanged into a pH 7.5 digestion buffer (0.1 M Tris or 0.1 M ammonium bicarbonate) using a NAP-5 column (GE Healthcare, Piscataway, NJ, USA). Lyophilized trypsin was dissolved in water to a final concentration of 1 mg/ml. Digestion was started with the addition of the 1-mg/ml trypsin solution to the reduced, alkylated, and buffer-exchanged Antibody 1 samples to achieve a 1:25 enzyme/substrate ratio. Digestion was carried out at 37° C. for 30 min. The final digest was quenched with the addition of 5 tl of 20% FA. LC-MS/MS peptide mapping analysis of the digested antibody samples was performed on an Agilent 1290 UHPLC system connected to a Thermo Scientific Q-Exactive Biopharma mass spectrometer as described in (Ren et al., Anal. Biochem. 392:12-21, 2009). Acquired LC-MS/MS raw data and sequences of AMG157 were used to identify and quantify modifications by MassAnalyzer software (Zhang, Anal. Chem. 81: 8354-8364 (2009)).

Surface plasmon resonance (SPR) is one of the traditional approaches that can measure the binding affinity for binding complexes. Binding complex with Kd<10⁻⁶M can be detected using SPR. By contrast, SEC binding affinity experiments can measure the antibody/ligand complex with Kd<10⁻⁸M. Weaker bound complexes (with Kd>10⁻⁸M) dissociate on the SEC column. As a result, the antibody and ligand molecules elute separately as unbound species.

50° C. one week (1W) stress of tezepelumab produced a very large percentage (˜67%) of high molecular weight (HMW) species. HMW species contained a high percentage of chemical modifications, including isomerization and deamidation on several residues, especially isomerization of LC D91. D91 isomerization was dramatically increased to ˜23% in HMW fraction versus 1% in monomer.

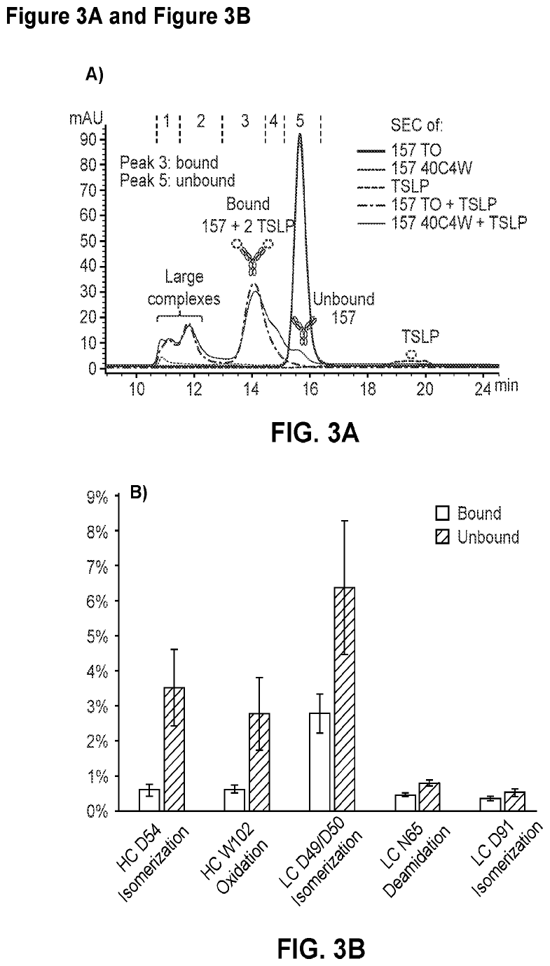

The impact of 4-week stress at 40° C. for four weeks (40C4W) on tezepelumab binding to TSLP was also assessed. By using SEC affinity binding followed by peptide mapping, five attributes (e.g., chemical modifications) of AMG 157 potentially impacting TSLP binding were chosen for analysis: HC D54 isomerization, HC W102 oxidation, LC D49 or D50 isomerization, LC N65 deamidation, and LC D91 isomerization (FIG. 3). For the D49 or D50 pair, it was difficult to distinguish the impact on binding between the two residues as both of them contribute to binding. The impact on binding was in the following order D54>W102>D49/D50>N65>D91. Only one of the modifications (LC D49 or D50 isomerization) may exceed 2% detectable level after tested end of shelf life conditions, which was established as 2 years at 5° C. followed by 2 months at 25° C. (2Y5C+2M25C). The binding became weaker than Kd=10⁻⁸ M from a typical antibody-antigen equilibrium dissociation constant of Kd=10⁻¹⁰ M, leading to dissociation of antibody-antigen complex on an SEC column and separate elution of the two molecules. The method showed good correlation to the traditional method of characterization of CEX fractions, which revealed that HC D54 isomerization strongly correlates with loss of potency as measured by the cell-based assay.

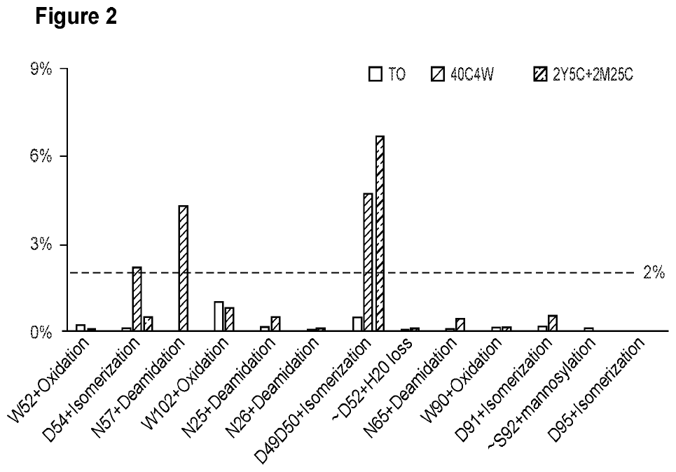

To verify peptide mapping method suitability, labile residues in CDRs and adjacent regions have been predicted in silico, suggesting that 16 modifications (attributes) of tezepelumab (AMG157) will likely take place and may impact the binding of AMG 157 to TSLP. Peptide mapping was performed on AMG 157 T0 and 40C4W samples and 16 predicted modifications were identified. HC D62 containing peptide had poor recovery, and HC D62 isomerization could not be quantified reliably. The peptide mapping results confirmed that the peptide mapping method can detect all of the modifications, except HC D62, and is suitable for the study (FIG. 2).

Several mutations of possible antibody attributes (residues with chemical modifications and following residues) were proposed to enhance the room-temperature stability, including HC D54E, HC G55A, LC D49E or D50E, LC S51A. U.S. Pat. No. 7,982,016 discloses an anti-TSLP antibody as sequence A5 (and also as chains H5, L5), which are set out in CDRs SEQ ID NO: 3-8.

50C1W stress of tezepelumab produced a very large percentage (˜67%) of HMW species. HMW species contained a high percentage of chemical modifications, including isomerization and deamidation on several residues, especially isomerization of LC D91 and HC D54, suggesting that HMW fraction may have lower potency due to the chemical modifications (attributes) impacting binding. Also, HMW species of tezepelumab 40C4W sample remained the same after binding, suggesting it was not involved in TSLP binding.

### Results

In total, 15 modifications are considered as potential modifying attributes in binding of AMG 157 to TSLP based on in silico sequence analysis (FIG. 1). Peptide mapping was applied to measure the percentage of the predicted attributes in AMG 157 T0 and 40° C.4W samples (FIG. 2). 40° C.4W corresponds to shelf life of liquid formulation (4° C.2Y) and is considered as reasonable condition of production and storage. Modifications >2% under reasonable conditions of production and storage are HC D54 isomerization, HC N57 deamidation, HC D62 isomerization, and LC D49D50 isomerization, however not all of these modifications impact binding to TSLP.

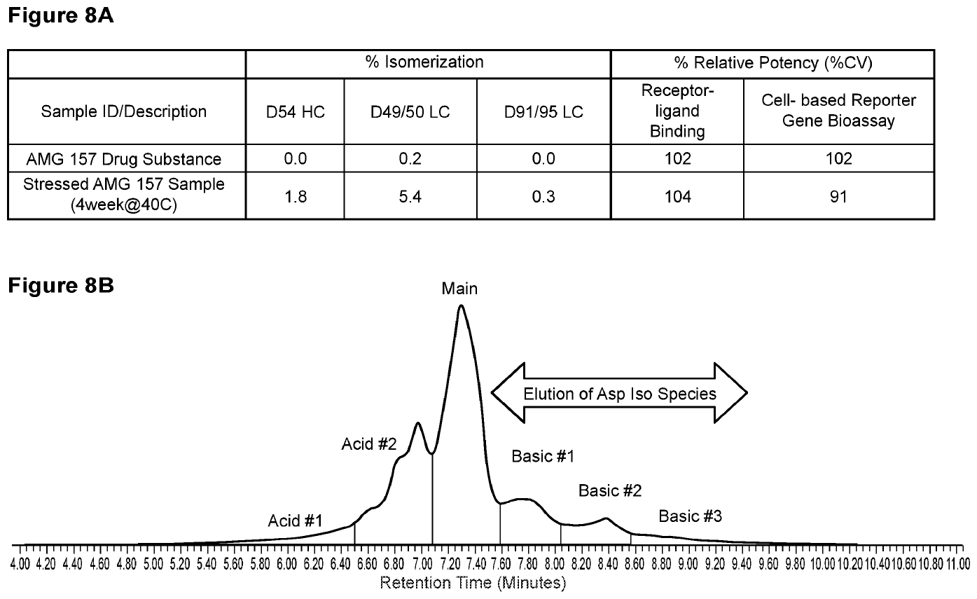

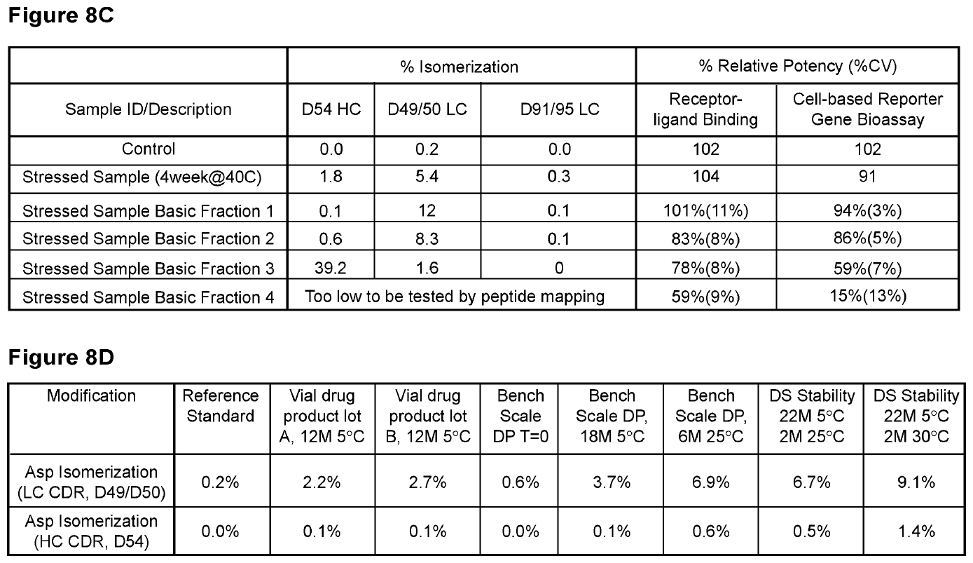

Chemical modifications of AMG 157 impacting binding to TSLP: SEC affinity binding of AMG157 40° C.4W and TSLP was used to experimentally determine the residues and modifications affecting the binding. The SEC-UV profiles suggest that after the 40° C.4W stress, unbound AMG157 eluting at 15.5 minutes constitutes less than 10%, indicating that loss of potency should be less than 10%, which agrees well with the potency measurements (FIG. 8A). Based on the elution time and theoretical molecular weight of AMG 157 (147 kDa) and TSLP (16 kDa), the peak eluting at 14 minutes was assigned as a complex containing 1 antibody and 2 TSLT molecules, the peak at 15 minutes as containing 1 antibody and 1 TSLP molecules. Other experiments using the same SEC conditions and multi-angle light scattering (MALS) detector identified similar in mass complexes eluting at these times. Large complexes eluting at ˜10.5 to 12.5 minutes were also observed when AMG 157 T0 or 40° C.4W binds to TSLP (FIG. 3A). They can be explained by the fact that TSLP contained 23% dimeric and 3% tetrameric species (see Materials and Methods section), which can potentially crosslink several antibodies, leading to larger complexes. It can be noted that biological characterization of CEX basic fractions revealed decreased receptor-ligand binding and cell-based reporter gene potency (FIG. 8C). Biochemical characterization (including peptide mapping) identified CDR Aspartic Acid Isomerization and several other modifications enriched in the basic CEX fractions including aggregation of fragmented species (HMW), partially reduced species, high mannose and afucosylated glycans, non-CDR Met oxidation, heavy chain C-terminal lysine and N-terminal signaling peptide, disulfide isoform A. The method comprising SEC of antibody-antigen was utilized as an orthogonal approach to assess and distinguish chemical modifications of the antibody impacting binding to TSLP from the modifications not impacting binding.

SEC of AMG157 40° C.4W and TSLP complex followed by LC-MS/MS characterization determined the ratio of modifications in unbound (5) and bound (3) fractions of AMG157. With statistical significance p<0.03, five residues were considered as relevant attributes: HC D54 isomerization, HC W102 oxidation, LC D49 or D50 isomerization, LC N65 deamidation, and LC D91 isomerization. For the D49D50 pair, it was difficult to distinguish impact between which of the two residues is mutated, both of them contribute to binding. Relative abundance of each modification is summarized in FIG. 3B. All the five attributes are below 10% in the bound and unbound fractions of AMG 157. The largest difference (ratio or fold-change) in the modification percentages between the bound and unbound fractions is found in HC D54 isomerization of AMG 157. The abundance of HC D54 isomerization in the bound and unbound fractions are ˜0.5% and ˜3.5%, respectively. LC D49D50 isomerization displays the highest percentage (˜6.2%) in the unbound fractions of AMG 157 40° C.4W.

Aspartate (D) is susceptible to isomerization in the current DG motif in HCDR2, but less so a different configuration if one of the residues is changed, e.g., G to A or D to E. Similarly, LCDR2 has the motif DDSDRPS, in which the aspartate(s) is susceptible to isomerization, so changes to the residues are proposed to improve stability, e.g., D to E or S to A.

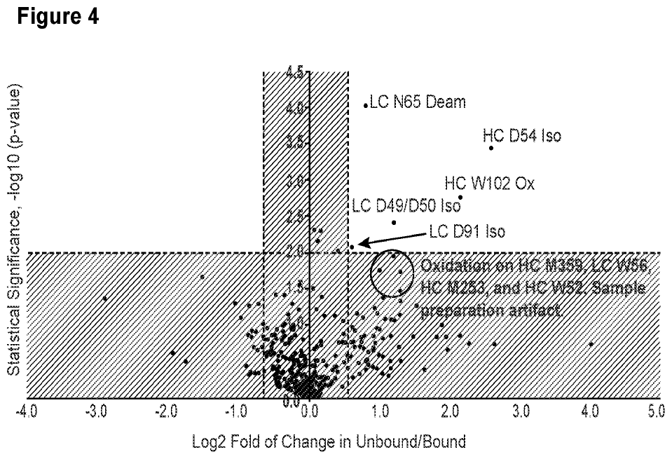

FIG. 4 shows the volcano plot for determining the relevant attributes in AMG157 40° C.4W binding to TSLP. In the statistical plot, log 2 (unbound/bound) indicates the strength of binding, which is D54>W102>D49D50>N65>D91. It was estimated that the equilibrium dissociation constant (Kd) of unbound AMG 157 to TSPL became Kd>10⁻⁸ M, at which point the degraded antibody was dissociated on column from TSLP. The unbound AMG157 demonstrated a much weaker binding as compared to the typical AMG157 Kd in nM range. In the SEC affinity binding method, D54 isomerization showed the highest fold change of unbound %/bound % (a value of 6) with high confidence of identification (p value=4×10−4). Combined with attribute modeling of AMG 157, SEC affinity binding experiments indicated that several mutations of relevant attributes (residues with chemical modifications and following residues) could enhance the room-temperature stability, including HC D54E, HC G55A, LC D49D50E, and LC S51A (FIG. 1, bottom panel).

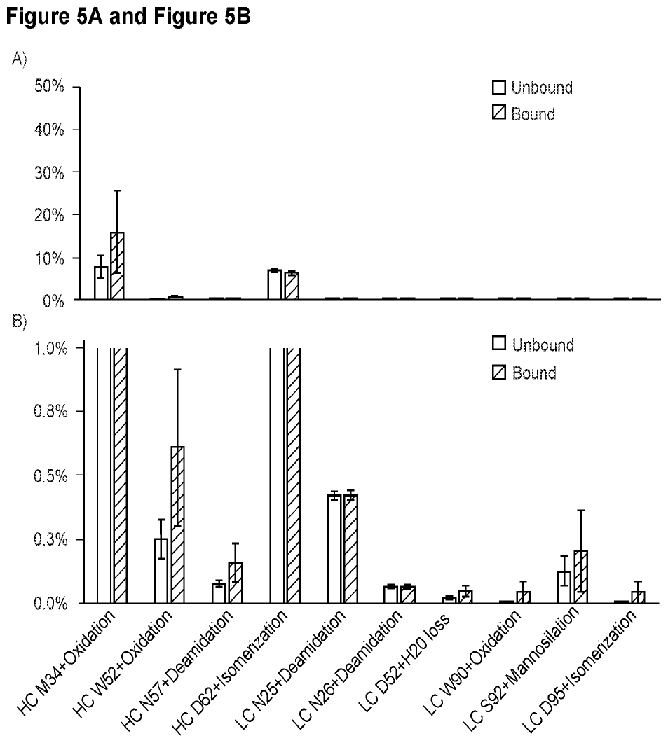

The residues and modifications exhibiting potential impact on binding (HC D54 isomerization, HC W102 oxidation, LC D49D50 isomerization, LC N65 deamidation, and LC D91 isomerization) were listed as possible attributes. On the other hand, several other residues considered as possible attributes did not impact binding by the SEC affinity method. FIG. 5 summarizes the relative abundance of the bound and unbound fraction in AMG157 40° C.4W for 11 modifications that did not change statistically significantly between the bound and unbound fractions (FIGS. 1 and 4). Also, except for HC M34 oxidation and HC D62 isomerization, all the modification percentages are below 1% after 4-week stress at 40° C. in formulation, indicating that they will not constitute significant percentage of modifications.

The attributes in the unbound antibody eluting at 15.6 minutes (peak 5) versus the complexes eluting at 10.5-12.5 minutes (peaks 1+2) were the same as in peak 5 versus peak 3, but with poorer statistics. This indicates that large complexes are antibody-TSLP complexes.

The present findings agree with the crystal structure of AMG 157 Fab binding to TSLP, suggesting that HC D54, HC W102 and LC D49 are in close distance with TSLP (within 6 Å) and very likely to engaged in binding directly. It should be mentioned that isoaspartate formation (HC D54, LC D49D50, D91) leads to elongation of the backbone (and shortening side chain), which changes position and orientation of these and nearby residues. This may lead to loss of binding. The closest atoms in the complex were selected to measure the distances without consideration for possible nature of interaction (hydrophobic, hydrogen bond, salt bridge). To summarize, long-range, allosteric effects may take place after isomerization at LC D49D50, LC N65, and LC D91, leading to loss of binding to TSLP.

Correlation to traditional approach: Following the traditional approach, AMG 157 40° C.4W sample was separated by CEX on main and three basic fractions, which were collected and characterized for chemical modifications by peptide mapping and for relative potency. Relative potency was measured by cell-based assay and binding assay. The results of the assays indicated that Basic Fraction 3 contained 39% of D54 isomerization and its cell-based potency was only 61%, indicating that this chemical modification impacts potency in this cell-based assay. This result is in a good agreement with the SEC affinity binding to TSLP, which identified HC D54 isomerization having the highest ratio in unbound versus bound fractions and impacting the binding the most.

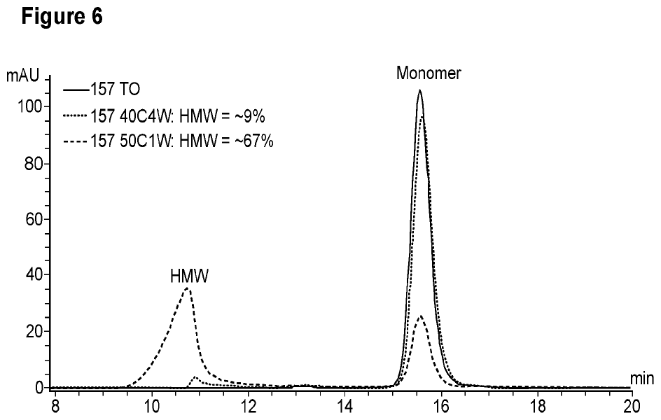

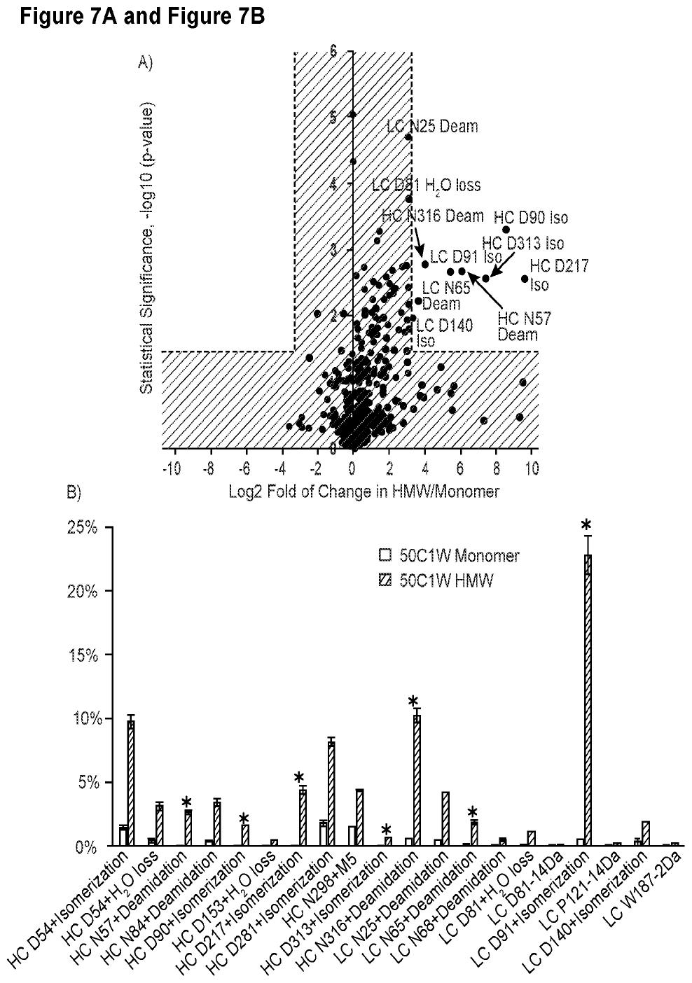

Chemical modifications correlating with aggregation at 50° C.: The percentage of HMW species was ˜9% in AMG157 40° C.4W sample. 50° C.1W stress of AMG157 produced a very large percentage (˜67%) of HMW species, which suggested partial unfolding of the antibody molecule at this temperature. Upon the separation by SEC, the HMW and monomer species in AMG157 50° C.1W were collected and analyzed using peptide mapping. By using the similar statistical approach employed in the SEC affinity binding measurement, a volcano plot was generated to assess the attributes involved in the formation of HMW species of AMG 157 (FIG. 6A). In the top right corner of the volcano plot, seven attributes appear to be the relevant for HMW formation with statistical significance (marked by asterisk in FIG. 6B). Abundances of these and several other modifications from “the gray area with near statistical significance” were plotted for HMW and monomer (FIG. 6B). Isomerization, deamidation and succinimide formation (H₂O loss) constitute the majority of the modifications strongly correlating to HMW formation. For example, LC D91 isomerization dramatically increased to ˜23% in HMW fraction versus 1% in monomer. Another modification potentially impacting binding, HC D54 isomerization was at 10% in HMW fraction versus 2% in monomer. That is, LC D91 isomerization and HC D54 isomerization each correlated with the formation of HMW species. The high levels of chemical modifications impacting binding in HMW fraction suggest that it may have lower potency as compared to the monomer. Also, HMW species of AMG 157 eluting from SEC at 10.5 minutes remained “unconsumed” after binding to TSLP, further suggesting that HMW species have weak binding. It is noted that AMG 157 40C4W HMW species are large in size and elute at the extreme of the size separation.

Suspected partial unfolding and observed dramatic HMW species formation in AMG157 after 50° C1W probably exposed residues that are not exposed and modified in a typical process. Utilization of the 40° C4W stressed materials, which produces less HMW species and modifications, should be more representative of typical process.

Overall, SEC affinity binding method experimentally determined residues and modifications affecting the binding of AMG 157 to TSLP. When statistical significance is p<0.03, HC D54 isomerization, HC W102 oxidation, LC D49 or D50 isomerization, LC N65 deamidation, and LC D91 isomerization appear to be relevant attributes of AMG157 binding to TSLP. Modifications impacting binding with high statistical significance and larger than 2% after 40° C.4W are HC D54 and LC D50 isomerization. It is noted that D49/50 isomerization does not correlate with loss in potency in other studies, such as biological assays, and the present finding under high stress conditions may be an artifact of the method. Chemical modifications correlate to the formation of HMW species after 50° C.1W stress, especially LC D91 isomerization. In view of these results, several mutations of AMG157 attributes (residues with chemical modifications and following residues) were proposed to improve room-temperature stability, including HC D54E, HC G55A, LC D49E or LC D50E, LC S51A.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain many equivalents to the specific embodiments of the invention described herein. Such equivalents are intended to be encompassed by the following claims.

What is claimed:

1. An anti-TSLP immunoglobulin, antigen binding protein or fragment thereof, or antibody or fragment thereof comprising
(A) a light chain variable domain comprising:
(i) a light chain CDR1 sequence comprising the amino acid sequence set forth in SEQ ID NO:3;
(ii) a light chain CDR2 sequence comprising the amino acid sequence set forth in SEQ ID NO:4; and
(iii) a light chain CDR3 sequence comprising the amino acid sequence set forth in SEQ ID NO:5; and
(B) a heavy chain variable domain comprising:
(i) a heavy chain CDR1 sequence comprising the amino acid sequence set forth in SEQ ID NO:6;
(ii) a heavy chain CDR2 sequence comprising an amino acid sequence with a mutation at one of the following residues, D54 or G55 set forth in SEQ ID NO:7, and
(iii) a heavy chain CDR3 sequence comprising the amino acid sequence set forth in SEQ ID NO: 8.

2. The anti-TSLP immunoglobulin, antigen binding protein or fragment thereof, or antibody or fragment thereof of claim 1, wherein the mutation in HCDR2 is D54E.

3. The anti-TSLP immunoglobulin, antigen binding protein or fragment thereof, or antibody or fragment thereof of claim 1, wherein the mutation in HCDR2 is G55A.

4. The anti-TSLP immunoglobulin, antigen binding protein or fragment thereof, or antibody or fragment thereof of any one of claims 1 to 3, wherein the HCDR2 has the sequence VIWYX₁X₂SNKHYADSVKG, wherein X₁ is D or E and X₂ is G or A (SEQ ID NO: 13), optionally the HCDR2 has the following sequence: VIWYEGSNKHYADSVKG (SEQ ID NO: 14), VIWYDASNKHYADSVKG (SEQ ID NO: 15) or VIWYEASNKHYADSVKG (SEQ ID NO: 16).

5. The anti-TSLP immunoglobulin, antigen binding protein or fragment thereof, or antibody or fragment thereof of any one of claims 1 to 4 optionally comprising a mutation in at least one of the following residues of LCDR2 D49, D50, or S51 of SEQ ID NO: 4.

6. The anti-TSLP immunoglobulin, antigen binding protein or fragment thereof, or antibody or fragment thereof of claim 5, wherein the mutation is any one of D49E, D50E, and/or S51A.

7. The anti-TSLP immunoglobulin, antigen binding protein or fragment thereof, or antibody or fragment thereof of claim 5 or 6, wherein the LCDR2 has the sequence X₁X₂X₃DRPS, wherein X₁ is D or E, X₂ is D or E, and X₃ is S or A (SEQ ID NO: 17), optionally the LCDR2 has the following sequence: EDSDRPS (SEQ ID NO: 18), DESDRPS (SEQ ID NO: 19), EESDRPS (SEQ ID NO: 20), DDADRPS (SEQ ID NO: 21), DEADRPS (SEQ ID NO: 22), EDADRPS (SEQ ID NO: 23) or EEADRPS (SEQ ID NO: 24).

8. An anti-TSLP immunoglobulin, antigen binding protein or fragment thereof, or antibody or fragment thereof comprising
(A) a light chain variable domain comprising:
(i) a light chain CDR1 sequence comprising the amino acid sequence set forth in SEQ ID NO:3;
(ii) a light chain CDR2 sequence comprising an amino acid sequence with a mutation in at least one of the following residues D49, D50, or S51 of SEQ ID NO: 4; and
(iii) a light chain CDR3 sequence comprising the amino acid sequence set forth in SEQ ID NO:5; and
(B) a heavy chain variable domain comprising:
(i) a heavy chain CDR1 sequence comprising the amino acid sequence set forth in SEQ ID NO:6;
(ii) a heavy chain CDR2 sequence comprising the amino acid sequence set forth in SEQ ID NO:7 and
(iii) a heavy chain CDR3 sequence comprising the amino acid sequence set forth in SEQ ID NO: 8.

9. The anti-TSLP immunoglobulin, antigen binding protein or fragment thereof, or antibody or fragment thereof of claim 8 wherein the mutation in LCDR2 is D49E, D50E and/or S51A.

10. The anti-TSLP immunoglobulin, antigen binding protein or fragment thereof, or antibody or fragment thereof of claim 8 or 9 wherein the mutation in LCDR2 is D49E.

11. The anti-TSLP immunoglobulin, antigen binding protein or fragment thereof, or antibody or fragment thereof of claim 8 or 9 wherein the mutation in LCDR2 is D50E.

12. The anti-TSLP immunoglobulin, antigen binding protein or fragment thereof, or antibody or fragment thereof of claim 8 or 9 wherein the mutation in LCDR2 is S51A.

13. The anti-TSLP immunoglobulin, antigen binding protein or fragment thereof, or antibody or fragment thereof of any one of claims 8 to 12, wherein the LCDR2 has the sequence X₁X₂X₃DRPS, wherein X₁ is D or E, X₂ is D or E, and X₃ is S or A (SEQ ID NO: 17), optionally the LCDR2 has the following sequence: EDSDRPS (SEQ ID NO: 18), DESDRPS (SEQ ID NO: 19), EESDRPS (SEQ ID NO: 20), DDADRPS (SEQ ID NO: 21), DEADRPS (SEQ ID NO: 22), EDADRPS (SEQ ID NO: 23) or EEADRPS (SEQ ID NO: 24).

14. The anti-TSLP immunoglobulin, antigen binding protein or fragment thereof, or antibody or fragment thereof of any one of claims 8 to 13, optionally comprising a mutation at one or more of the following residues D54 or G55 in HCDR2 set out in SEQ ID NO: 7.

15. The anti-TSLP immunoglobulin, antigen binding protein or fragment thereof, or antibody or fragment thereof of claim 14, wherein the mutation is any one of D54E and/or G55A in HCDR2 set out in SEQ ID NO: 7.

16. The anti-TSLP immunoglobulin, antigen binding protein or fragment thereof, or antibody or fragment thereof of claim 14 or 15, wherein the HCDR2 has the sequence VIWYX₁X₂SNKHYADSVKG, wherein X₁ is D or E and X₂ is G or A (SEQ ID NO: 13), optionally the HCDR2 has the following sequence: VIWYEGSNKHYADSVKG (SEQ ID NO: 14), VIWYDASNKHYADSVKG (SEQ ID NO: 15) or VIWYEASNKHYADSVKG (SEQ ID NO: 16).

17. The anti-TSLP immunoglobulin, antigen binding protein or fragment thereof, or antibody or fragment thereof of any one claims 1-16 comprising:
(A) a light chain variable domain selected from the group consisting of:
i. a sequence of amino acids at least 80% identical to SEQ ID NO: 12;
ii. a sequence of amino acids encoded by a polynucleotide sequence that is at least 80% identical to SEQ ID NO: 11; or
iii. a sequence of amino acids encoded by a polynucleotide that hybridizes under moderately stringent conditions to the complement of a polynucleotide consisting of SEQ ID NO: 11; or
(B) a heavy chain variable domain selected from the group consisting of:
i. a sequence of amino acids that is at least 80% identical to SEQ ID NO: 10;
ii. a sequence of amino acids encoded by a polynucleotide sequence that is at least 80% identical to SEQ ID NO: 9; or
iii. a sequence of amino acids encoded by a polynucleotide that hybridizes under moderately stringent conditions to the complement of a polynucleotide consisting of SEQ ID NO: 9; or
(C) a light chain variable domain of (A) and a heavy chain variable domain of (B),
wherein the anti-TSLP immunoglobulin, antigen binding protein or fragment thereof, or antibody or fragment thereof retains the CDRs of the anti-TSLP antigen binding proteins or fragment thereof.

18. The antigen binding protein or fragment thereof of any one of claims 1 to 17, wherein the anti-TSLP immunoglobulin, antigen binding protein or fragment thereof, or antibody or fragment thereof comprises a heavy chain comprising the amino acid sequence of SEQ ID NO: 10 or SEQ ID NO: 25-28, a light chain comprising the amino acid sequence of SEQ ID NO: 12 or SEQ ID NO: 29-36.

19. The anti-TSLP immunoglobulin, antigen binding protein or fragment thereof, or antibody or fragment thereof of any one of claims 1 to 18 wherein the anti-TSLP immunoglobulin, antigen binding protein or fragment thereof, or antibody or fragment thereof is selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgM antibody, an IgG1 antibody, an IgG2 antibody, an IgG3 antibody, and an IgG4 antibody.

20. The anti-TSLP immunoglobulin, antigen binding protein or fragment thereof, or antibody or fragment thereof of any one of claims 1 to 18, wherein the antigen binding protein is a human antibody.

21. The antibody of claim 19 or 20 that is an IgG2 antibody.

22. The immunoglobulin, antigen binding protein or fragment thereof, or antibody or fragment thereof of any one of claims 1 to 21, wherein the anti-TSLP antigen binding protein or fragment thereof specifically binds to a TSLP polypeptide as set forth in amino acids 29-159 of SEQ ID NO: 2.

23. The anti-TSLP immunoglobulin, antigen binding protein or fragment thereof, or antibody or fragment thereof of any one of claims 1 to 22, wherein both binding sites of the anti-TSLP antigen binding protein or fragment thereof have identical binding to TSLP.

24. The anti-TSLP immunoglobulin, antigen binding protein or fragment thereof, or antibody or fragment thereof of any one of claims 1 to 23 that binds TSLP with an affinity that is numerically no more than 10⁻⁸ M Kd.

25. A composition comprising the anti-TSLP immunoglobulin, antigen binding protein or fragment thereof, or antibody or fragment thereof of any one of claims 1 to 24 and a pharmaceutically acceptable carrier, excipient or diluent.

26. An isolated nucleic acid comprising a polynucleotide sequence encoding the light chain variable domain, the heavy chain variable domain, or both, of the immunoglobulin, antigen binding protein or fragment thereof, or antibody or fragment thereof of any one of claims 1 to 24.

27. A recombinant expression vector comprising the nucleic acid of claim 26.

28. A host cell comprising the vector of claim 27.

29. A method of producing an immunoglobulin, antigen binding protein or fragment thereof, or antibody or fragment thereof that specifically binds to a TSLP polypeptide comprising amino acids 29-159 of SEQ ID NO: 2, comprising incubating the host cell of claim 28 under conditions that allow it to express the immunoglobulin, antigen binding protein or fragment thereof, or antibody or fragment thereof, wherein said host cell comprises (i) a recombinant expression vector encoding the light chain variable domain of the immunoglobulin, antigen binding protein or fragment thereof, or antibody or fragment thereof of any one of claims 1-24 and a recombinant expression vector encoding the heavy chain variable domain of the immunoglobulin, antigen binding protein or fragment thereof, or antibody or fragment thereof of any one of claims 1-24, or (ii) a recombinant expression vector encoding both the light chain variable domain and the heavy chain variable domain of the immunoglobulin, antigen binding protein or fragment thereof, or antibody or fragment thereof of claims 1 to 24.

30. The anti-TSLP immunoglobulin, antigen binding protein or fragment thereof, or antibody or fragment thereof of any one of claims 1 to 24, having increased stability at 25° C. compared to an anti-TSLP immunoglobulin, antigen binding protein or fragment thereof, or antibody or fragment thereof having the amino acid sequences set out in SEQ ID NO: 10 and SEQ ID NO: 12.

31. The anti-TSLP immunoglobulin, antigen binding protein or fragment thereof, or antibody or fragment thereof of any one of claims 1 to 24, having increased stability at 40° C. after 4 weeks compared to an anti- immunoglobulin, antigen binding protein or fragment thereof, or antibody or fragment thereof having the amino acid sequences set out in SEQ ID NO: 10 and SEQ ID NO: 12.

32. The anti-TSLP immunoglobulin, antigen binding protein or fragment thereof, or antibody or fragment thereof of any one of claims 1 to 24, having decreased high molecular weight species at 40° C. after 4 weeks compared to an anti-TSLP immunoglobulin, antigen binding protein or fragment thereof, or antibody or fragment thereof having the amino acid sequences set out in SEQ ID NO: 10 and SEQ ID NO: 12.

33. The anti-TSLP immunoglobulin, antigen binding protein or fragment thereof, or antibody or fragment thereof of any one of claims 1 to 24, wherein the anti-TSLP immunoglobulin, antigen binding protein or fragment thereof, or antibody or fragment thereof had decreased isomerization at 50° C. compared to an anti-TSLP immunoglobulin, antigen binding protein or fragment thereof, or antibody or fragment thereof having the amino acid sequences set out in SEQ ID NO: 10 and SEQ ID NO: 12.

34. The anti-TSLP immunoglobulin, antigen binding protein or fragment thereof, or antibody or fragment thereof of any one of claims 1 to 24, wherein less than 2% of the anti-TSLP immunoglobulin, antigen binding protein or fragment thereof, or antibody or fragment thereof shows isomerization and/or deamidation after at least 2 weeks (optionally, after at least 1 month, after at least 2 months, after at least 3 months, after at least 4 months, after at least 5 months or after at least 6 months) of storage at about 25° C., as determined by SEC.

35. The anti-TSLP immunoglobulin, antigen binding protein or fragment thereof, or antibody or fragment thereof of any one of claims 1 to 24, wherein less than 2% of the immunoglobulin, antigen binding protein or fragment thereof, or antibody or fragment thereof shows isomerization and/or deamidation after about 22 months to about 36 months of storage at 2° C. to 8° C. followed by at least 2 weeks or at least 1 month or at least 2 months of storage at about 25° C., as determined by SEC.

36. A method for treating an inflammatory disease in a subject comprising administering to the subject a therapeutically effective amount of the immunoglobulin, antigen binding protein or fragment thereof, or antibody or fragment thereof of any one of the claims 1-24 or a composition of claim 25.

37. The method of claim 36, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).

38. The method of claim 36 or 37, comprising administering the composition at an interval of every 2 weeks or every 4 weeks.

39. The method of any one of claims 36 to 38, wherein the composition is administered for a period of at least 4 months, 6 months, 9 months, 1 year or more.

40. The method of any one of claims 37 to 39, wherein the asthma is severe asthma.

41. The method of any one of claims 37 to 40, wherein the asthma is eosinophilic or non-eosinophilic asthma.

42. A method of making a composition comprising a plurality of anti-TSLP monoclonal antibodies or antigen binding fragments thereof each comprising:
a light chain CDR1 sequence comprising the amino acid sequence set forth in SEQ ID NO:3;
a light chain CDR2 sequence comprising the amino acid sequence set forth in SEQ ID NO:4;
a light chain CDR3 sequence comprising the amino acid sequence set forth in SEQ ID NO:5;
a heavy chain CDR1 sequence comprising the amino acid sequence set forth in SEQ ID NO:6;
a heavy chain CDR2 sequence comprising the amino acid sequence set forth in SEQ ID NO: 7; and
a heavy chain CDR3 sequence comprising the amino acid sequence set forth in SEQ ID NO: 8,
the method comprising enriching the composition for IgG2 anti-TSLP monoclonal antibodies or antigen binding fragments thereof for at least one of the following attributes:
L-aspartate at HC position 54, relative to isoAspartate (isoAsp) or cyclic aspartate (cAsp) at HC position 54;
non-oxidized HC W102, relative to oxidized HC W102;
L-aspartate at LC position 49 or position 50, relative to isoAsp or cAsp at LC position 49 or position 50;
LC N65 relative to deamidated LC N65; or
L-aspartate at LC position 91, relative to isoAsp or cAsp at LC position 91.

43. The method of claim 42 wherein no more than 0.9% of the anti-TSLP monoclonal antibodies comprise isomerized HC D54.

44. The method of claim 42 or 43 wherein no more than 2% of the anti-TSLP monoclonal antibodies comprise oxidized HC W102.

45. The method of any one of claims 42 to 44, wherein no more than 0.9% of the anti-TSLP monoclonal antibodies comprise isomerized LC D50 or LC D49.

46. The method of any one of claims 42 to 45, wherein no more than 0.5% of the anti-TSLP monoclonal antibodies comprise deamidated LC N65.

47. The method of any one of claims 42 to 46, wherein no more than 0.9% of the anti-TSLP monoclonal antibodies comprise isomerized LC D91.

48. The method of any one of claims 42 to 47, wherein the anti-TSLP antibody is an IgG2 antibody.

49. The method of any one of claims 42 to 48, wherein the anti-TSLP antibody comprises (i) L-aspartate at HC D54 and (ii) L-aspartate at LC D49 and/or D50.

50. The method of any one of claims 42 to 49, wherein the anti-TSLP antibody is enriched in L-aspartate at HC D54 to at least 6-fold over the levels of isoAsp.

51. The method of any one of claims 42 to 50, wherein the anti-TSLP antibody comprises a heavy chain variable region set out in SEQ ID NO: 10 and a light chain variable region set out in SEQ ID NO: 12.

52. A composition comprising anti-TSLP monoclonal antibodies each comprising:
a light chain CDR1 sequence comprising the amino acid sequence set forth in SEQ ID NO:3;
a light chain CDR2 sequence comprising the amino acid sequence set forth in SEQ ID NO:4;
a light chain CDR3 sequence comprising the amino acid sequence set forth in SEQ ID NO:5;
a heavy chain CDR1 sequence comprising the amino acid sequence set forth in SEQ ID NO:6;
a heavy chain CDR2 sequence comprising the amino acid sequence set forth in SEQ ID NO: 7; and
a heavy chain CDR3 sequence comprising the amino acid sequence set forth in SEQ ID NO: 8,
the composition comprising a limited content of isomerized HC D54 and/or a limited content of isomerized LC D49 or D50, effective for the anti-TSLP monoclonal antibodies of the composition to bind to TLSP with a Kd that is numerically less than or equal to 10⁻⁸ M.

53. A composition comprising IgG2 anti-TSLP monoclonal antibodies, each comprising a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ ID NO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO: 8,
wherein at least one of: no more than 0.9% of the anti-TSLP monoclonal antibodies comprise isomerized HC D54;
no more than 2% of the anti-TSLP monoclonal antibodies comprise oxidized HC W102;
no more than 0.9% of the anti-TSLP monoclonal antibodies comprise isomerized LCD49 or LC D50;
no more than 0.5% of the anti-TSLP monoclonal antibodies comprise deamidated LC N65; or
no more than 0.9% of the anti-TSLP monoclonal antibodies comprise isomerized LC D91.

54. The composition of claim 53 wherein no more than 0.9% of the anti-TSLP monoclonal antibodies comprise isomerized HC D54.

55. The composition of claim 53 or 54 wherein no more than 2% of the anti-TSLP monoclonal antibodies comprise oxidized HC W102.

56. The composition of any one of claims 53 to 55, wherein no more than 0.9% of the anti-TSLP monoclonal antibodies comprise isomerized LCD49 or LC D50.

57. The composition of any one of claims 53 to 56, wherein no more than 0.5% of the anti-TSLP monoclonal antibodies comprise deamidated LC N65.

58. The composition of any one of claims 53 to 57, wherein no more than 0.9% of the anti-TSLP monoclonal antibodies comprise isomerized LC D91.

59. The composition of any one of claims 53 to 58, wherein the anti-TSLP antibody is an IgG2 antibody.

60. The composition of any one of claims 53 to 59 wherein the anti-TSLP antibody comprises a combination of L-aspartate at HC 54 and L-aspartate at LC 49 and/or LC50.

61. The composition of any one of claims 4538 to 60, wherein the anti-TSLP antibody is enriched in L-aspartate at HC54 to at least 6-fold over the levels of isoAsp.

62. The composition of any one of claims 53 to 61 wherein the anti-TSLP antibody comprises a heavy chain variable region set out in SEQ ID NO: 10 and a light chain variable region set out in SEQ ID NO: 12 and
wherein at least one of: no more than 0.9% of the anti-TSLP monoclonal antibodies comprise isomerized HC D54;
no more than 2% of the anti-TSLP monoclonal antibodies comprise oxidized HC W102;
no more than 0.9% of the anti-TSLP monoclonal antibodies comprise isomerized LCD49 or LC D50;
no more than 0.5% of the anti-TSLP monoclonal antibodies comprise deamidated LC N65; or
no more than 0.9% of the anti-TSLP monoclonal antibodies comprise isomerized LC D91.

63. A composition comprising anti-TSLP monoclonal antibodies, each comprising a light chain CDR1 sequence comprising the amino acid sequence set forth in SEQ ID NO: 3; a light chain CDR2 sequence comprising the amino acid sequence set forth in SEQ ID NO: 4; a light chain CDR3 sequence comprising the amino acid sequence set forth in SEQ ID NO: 5; a heavy chain CDR1 sequence comprising the amino acid sequence set forth in SEQ ID NO: 6; a heavy chain CDR2 sequence comprising the amino acid sequence set forth in SEQ ID NO: 7; and a heavy chain CDR3 sequence comprising the amino acid sequence set forth in SEQ ID NO: 8, wherein at least one of:
greater than 98% of the anti-TSLP monoclonal antibodies of the composition comprise L-aspartate at HC position 54, relative to isoAsp or cAsp at HC position 54;
at least 99% of the anti-TSLP monoclonal antibodies of the composition comprise non-oxidized HC W102 relative to oxidized HC W102;
at least 97% of the anti-TSLP monoclonal antibodies of the composition comprise L-aspartate at LC position 49 or 50, relative to isoAsp or cAsp at LC position 49 or position 50;
at least 99.1% of the anti-TSLP monoclonal antibodies of the composition comprise LC N65 relative to deamidated LC N65; or
at least 99.1% of the anti-TSLP monoclonal antibodies of the composition comprise L-aspartate at LC position 91, relative to isoAsp or cAsp at LC position 91.
